(19) United States
(12) Patent Application Publication
(10) Pub. No.: US 2013/0072413 A1
(43) Pub. Date: Mar. 21, 2013

(54) DETERGENT COMPOSITIONS COMPRISING PRIMARY SURFACTANT SYSTEMS COMPRISING HIGHLY BRANCHED ISOPRENOID-BASED and OTHER SURFACTANTS

(71) Applicant: The Procter & Gamble Company, Cincinnati, OH (US)

(72) Inventors: Stephanie Ann Urbin, Liberty Township, OH (US); Sherri Lynn Randall, Hamilton, OH (US); Kenneth Nathan Price, Cincinnati, OH (US); Randall Thomas Reilman, Cincinnati, OH (US); Phillip Kyle Vinson, Fairfield, OH (US); Praveen Kumar Depa, Hyde Park, OH (US)

(73) Assignee: The Procter & Gamble Company, Cincinnati, OH (US)

(21) Appl. No.: 13/623,134

(22) Filed: Sep. 20, 2012

Related U.S. Application Data
(60) Provisional application No. 61/536,819, filed on Sep. 20, 2011.

(51) Int. Cl. C11D 3/60; B65D 85/00; A61K 8/18; A61Q 5/12; C11D 17/00; C11D 17/06

(52) U.S. Cl. 510/220; 510/108; 510/505; 510/506; 510/495; 510/503; 510/467; 510/470; 510/502; 510/494; 510/493; 510/500; 510/447; 510/337; 510/403; 510/235; 510/283; 510/445; 424/70.1; 510/515; 510/504; 206/525

(57) ABSTRACT

The present invention relates to detergent compositions containing a surfactant system comprising a highly branched surfactant as the primary surfactant. Specifically, the invention relates to detergent compositions containing a surfactant system comprising greater than about 25% of a highly branched surfactant.

### FIELD OF THE INVENTION

The present invention relates to detergent compositions containing a surfactant system comprising a highly branched surfactant as the primary surfactant. Specifically, the invention relates to detergent compositions containing a surfactant system comprising greater than about 25% of a highly branched surfactant.

### BACKGROUND OF THE INVENTION

Most conventional detergent compositions contain mixtures of various detersive surfactant components. Commonly encountered surfactant components include various anionic surfactants, especially the alkyl benzene sulfonates, alkyl sulfates, alkyl alkoxy sulfates and various nonionic surfactants, such as alkyl ethoxylates and alkylphenol ethoxylates. Surfactants have found use as detergent components capable of the removal of a wide variety of soils and stains. A consistent effort however is made by detergent manufacturers to improve detersive properties of detergent compositions by providing new and improved surfactants. Today, challenges facing detergent manufacturers include colder wash temperatures, less efficient builders, liquid or powder products without calcium control, and the desire to reduce surfactant use overall.

Highly branched surfactants of various types are known. For example, isoprenoid-based poly-branched detergent alcohols, including 4,8,12-trimethyltridecan-1-ol and 3-ethyl-7,11-dimethyldodecan-1-ol, and their surfactant derivatives, which may be derived from natural derived farnesene, farnesene obtained from genetically modified organisms, synthetically derived trimers of isoprene, or mixtures thereof, are known. Processes of making such detergent alcohols and surfactants are also known. The use of highly branched surfactants, including isoprenoid-based poly-branched surfactants, as low-level co-surfactants in detergent compositions is also known. Highly branched surfactants, however, have not been successfully formulated into detergent compositions at increased levels, namely at levels where the branched surfactant is the primary surfactant of the surfactant system.

As such, there is a need to formulate detergent compositions comprising surfactant systems containing a highly branched surfactant as the majority of the surfactant system, rather than as a low-level co-surfactant. Such detergent compositions provide superior benefits in cold water grease cleaning, cold water solubility, and neat product phase stability. It is believed that a highly branched surfactant exhibits advantageous packing at soil interfaces and superior resistance to calcium and/or magnesium ions (resulting in solubility benefits).

### SUMMARY OF THE INVENTION

This invention relates to a detergent composition comprising a surfactant system, where the surfactant system comprises greater than about 25% of one or more highly branched surfactants, one or more adjunct cleaning additives, and a carrier.

### DETAILED DESCRIPTION OF THE INVENTION

As used herein, the term “surfactant A+B”, “A and B”, or “A+B” refers to a blend of surfactant A and surfactant B (as defined below). For example, the term “A+B AE1.8S” refers to a mixture of surfactant A and surfactant B that has been derivatized into an alkyl ethoxy sulfate blend with an average of 1.8 mols of ethoxylation; likewise, the term “80A:20B amine oxide” refers to an 80:20 wt/wt mixture of surfactant A and surfactant B that has been derivatized into an amine oxide.

As used herein, the articles including “the”, “a” and “an” when used in a claim or in the specification, are understood to mean one or more of what is claimed or described.

As used herein, the terms “include”, “includes” and “including” are meant to be non-limiting.

As used herein, the terms “fabric”, “textile”, and “cloth” are used non-specifically and may refer to any type of flexible material consisting of a network of natural or artificial fibers, including natural, artificial, and synthetic fibers, such as, but not limited to, cotton, linen, wool, polyester, nylon, silk, acrylic, and the like, including blends of various fabrics or fibers.

As used herein, the phrase “detergent composition” includes compositions and formulations designed for treating, including cleaning, textiles, fabric, and hard surfaces. Such compositions include but are not limited to, laundry cleaning compositions and laundry detergents, fabric softening compositions, fabric enhancing compositions, fabric freshening compositions, laundry pre-wash compositions, laundry pre-treat compositions, laundry additives, a fabric treatment composition, a dry cleaning composition, a laundry soak or spray treatment, a laundry rinse additive, a wash additive, a post-rinse fabric treatment, an ironing aid, a liquid hand dishwashing composition, an automatic dishwashing detergent, and a hard surface cleaner. A detergent composition may be in the form of granules (e.g., powder), a liquid (including heavy duty liquid (“HDL”) detergents), a gel, a paste, a bar, a single-phase or a multi-phase unit dose composition, a detergent contained in a single-phase or multi-phase or multi-compartment water soluble pouch, a detergent contained on or in a porous substrate or nonwoven sheet, a flake formulation, a spray product, or a delayed delivery formulation. In the context of laundry, such compositions may be used as a pre-laundering treatment, a post-laundering treatment, or may be added during the rinse or wash cycle of the laundering operation.

### Detergent Composition

The present invention relates to a detergent composition comprising a surfactant system, where the surfactant system comprises greater than about 25% of one or more highly branched surfactants and, optionally, one or more linear or lightly branched surfactants; one or more adjunct cleaning additives; and a carrier.

Surfactant System

The detergent compositions of the present invention comprise from about 0.001 wt % to about 100 wt % of a surfactant system, in certain aspects, from about 0.1 wt % to about 80 wt % of a surfactant system or from about 1 wt % to about 25 wt % of a surfactant system.

The surfactant system of the present invention comprises greater than about 25%, in some aspects, greater than about 35%, in certain aspects, greater than about 50%, of one or more highly branched surfactants and, optionally, one or more linear or lightly branched surfactants.

Highly Branched Surfactants

The highly branched surfactants of the present invention comprise surfactants where the hydrophobe moiety of the surfactant compound has a main carbon chain having greater than about 2.4 alkyl branches pendant from the main chain.

In some aspects, the highly branched surfactant may include an isoprenoid-based surfactant of the structure

E-Y-Z

where E is one or more saturated, acyclic C10-C24 isoprenoid-based hydrophobe(s) and Y and Z are as defined below; such isoprenoid-based surfactants are exemplified by formulas i through xv below:

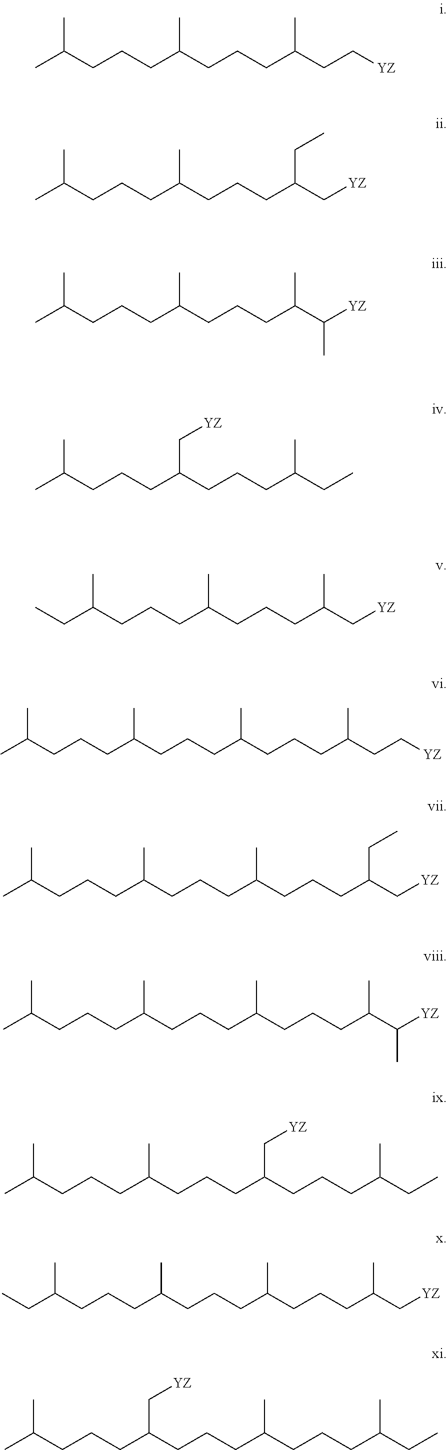

where Y is CH₂ or null and Z is selected such that the resulting surfactant is an alkyl carboxylate surfactant, an alkyl polyalkoxy surfactant, an alkyl anionic polyalkoxy sulfate surfactant, an alkyl glycerol ester sulfonate surfactant, an alkyl dimethyl amine oxide surfactant, an alkyl polyhydroxy based surfactant, an alkyl phosphate ester surfactant, an alkyl glycerol sulfonate surfactant, an alkyl polygluconate surfactant, an alkyl polyphosphate ester surfactant, an alkyl phosphonate surfactant, an alkyl polyglycoside surfactant, an alkyl monoglycoside surfactant, an alkyl diglycoside surfactant, an alkyl sulfosuccinate surfactant, an alkyl disulfate surfactant, an alkyl disulfonate surfactant, an alkyl sulfosuccinamate surfactant, an alkyl glucamide surfactant, an alkyl taurinate surfactant, an alkyl sarcosinate surfactant, an alkyl glycinate surfactant, an alkyl isethionate surfactant, an alkyl dialkanolamide surfactant, an alkyl monoalkanolamide surfactant, an alkyl monoalkanolamide sulfate surfactant, an alkyl diglycolamide surfactant, an alkyl diglycolamide sulfate surfactant, an alkyl glycerol ester surfactant, an alkyl glycerol ester sulfate surfactant, an alkyl glycerol ether surfactant, an alkyl glycerol ether sulfate surfactant, alkyl methyl ester sulfonate surfactant, an alkyl polyglycerol ether surfactant, an alkyl polyglycerol ether sulfate surfactant, an alkyl sorbitan ester surfactant, an alkyl ammonioalkanesulfonate surfactant, an alkyl amidopropyl betaine surfactant, an alkyl allylated quat based surfactant, an alkyl monohydroxyalkyl-di-alkylated quat based surfactant, an alkyl di-hydroxyalkyl monoalkyl quat based surfactant, an alkylated quat surfactant, an alkyl trimethylammonium quat surfactant, an alkyl polyhydroxalkyl oxypropyl quat based surfactant, an alkyl glycerol ester quat surfactant, an alkyl glycol amine quat surfactant, an alkyl monomethyl dihydroxyethyl quaternary ammonium surfactant, an alkyl dimethyl monohydroxyethyl quaternary ammonium surfactant, an alkyl trimethylammonium surfactant, an alkyl imidazoline-based surfactant, an alken-2-yl-succinate surfactant, an alkyl α-sulfonated carboxylic acid surfactant, an alkyl α-sulfonated carboxylic acid alkyl ester surfactant, an alpha olefin sulfonate surfactant, an alkyl phenol ethoxylate surfactant, an alkyl benzenesulfonate surfactant, an alkyl sulfobetaine surfactant, an alkyl hydroxysulfobetaine surfactant, an alkyl ammoniocarboxylate betaine surfactant, an alkyl sucrose ester surfactant, an alkyl alkanolamide surfactant, an alkyl di(polyoxyethylene)monoalkyl ammonium surfactant, an alkyl mono(polyoxyethylene)dialkyl ammonium surfactant, an alkyl benzyl dimethylammonium surfactant, an alkyl aminopropionate surfactant, an alkyl amidopropyl dimethylamine surfactant, or a mixture thereof; if Z is a charged moiety, Z is charge-balanced by a suitable metal or organic counter ion. Suitable counter ions include a metal counter ion, an amine, or an alkanolamine, e.g., C1-C6 alkanolammonium. More specifically, suitable counter ions include Na+, Ca+, Li+, K+, Mg+, e.g., monoethanolamine (MEA), diethanolamine (DEA), triethanolamine (TEA), 2-amino-1-propanol, 1-aminopropanol, methyldiethanolamine, dimethylethanolamine, monoisopropanolamine, triisopropanolamine, 1-amino-3-propanol, or mixtures thereof.

In some aspects, the highly branched surfactant is selected from surfactant A, surfactant B, or a mixture thereof, as defined below. “Surfactant A” or “A” is represented by the following formula:

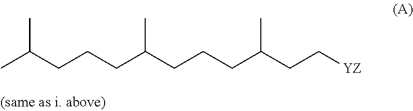

where Y and Z are as defined above. “Surfactant B” or “B” is represented by the following formula:

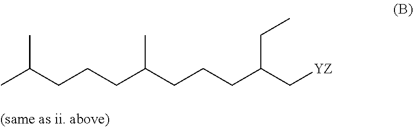

where Y and Z are as defined above.
In further aspects, the ratio by weight of “surfactant A” to “surfactant B” ranges from about 50:50 to about 97:5. In some aspects, the ratio of “surfactant A” to “surfactant B” ranges from about 50:50 to about 95:5 or from about 65:35 to about 80:20.

In some aspects, the highly branched surfactant may include a non-isoprenoid-based surfactant, such as those described in US Patent Application Nos. 2011/0171155A1 and 2011/0166370A1, where are hereby incorporated by reference.

Linear or Lightly Branched Surfactants

The surfactant system of the present invention may optionally comprise one or more linear or lightly branched surfactants. Lightly branched surfactants of the present invention comprise surfactants where the hydrophobe moiety of the surfactant compound has a main carbon chain with less than about 2.4 alkyl branches pendant from the main chain.

In some aspects, the optional linear or lightly branched surfactant is a linear surfactant. Linear surfactants derived from agrochemical oils are particularly useful for the present invention. Agrochemical oils that are typically used to produce naturally-derived surfactants (anionic surfactants, non-ionic surfactants, cationic surfactants, zwitterionic surfactants) include coconut oil, palm kernel oil, soybean oil, or other vegetable-based oils.

In some aspects, the linear or lightly branched surfactant comprises one or more surfactants that is typically utilized in detergent or cleaning compositions. Suitable such surfactants include anionic surfactants, zwitterionic surfactants, amphoteric surfactants, cationic surfactants, or combinations thereof. The concentration of such linear or lightly branched surfactant in the surfactant system of the composition may range from about 0% to about 50% of the surfactant system.

Suitable linear and lightly branched surfactants are anionic surfactants, including the C₁₀-C₁₅ alkyl benzene sulfonates (LAS), the linear or lightly branched alkyl and alkyl ether sulfates, water-soluble salts of organic, sulfuric acid reaction products, reaction products of fatty acids esterified with isethionic acid, succinates, olefin sulfonates having about 10 to about 24 carbon atoms, and beta-alkyloxy alkane sulfonates.

Still further nonlimiting examples of linear or lightly branched anionic surfactants useful herein include: C₁₀-C₂₀ primary or branched chain and random alkyl sulfates (AS); C₁₀-C₁₈ secondary (2,3)alkyl sulfates; C₁₀-C₁₈ alkyl alkoxy sulfates (AEₓS) wherein x is from 1-30; C10-C18 alkyl alkoxy carboxylates comprising 1-5 ethoxy units; mid-chain branched alkyl sulfates as discussed in U.S. Pat. No. 6,020,303 and U.S. Pat. No. 6,060,443; mid-chain branched alkyl alkoxy sulfates as discussed in U.S. Pat. No. 6,008,181 and U.S. Pat. No. 6,020,303; modified alkylbenzene sulfonate (MLAS) as discussed in WO 99/05243, WO 99/05242 and WO 99/05244; methyl ester sulfonate (MES); and alpha-olefin sulfonate (AOS).

Suitable anionic surfactants may be any of the conventional anionic surfactant types typically used in liquid detergent products. Such surfactants include the alkyl benzene sulfonic acids and their salts as well as alkoxylated or non-alkoxylated alkyl sulfate materials. Exemplary anionic surfactants are the alkali metal salts of C₁₀-C₁₆ alkyl benzene sulfonic acids, preferably C₁₁-C₁₄ alkyl benzene sulfonic acids. In one aspect, the alkyl group is linear. Such linear alkyl benzene sulfonates are known as “LAS”. Such surfactants and their preparation are described for example in U.S. Pat. Nos. 2,220,099 and 2,477,383. Especially useful are the sodium and potassium linear straight chain alkylbenzene sulfonates in which the average number of carbon atoms in the alkyl group is from about 10 to 15. Sodium C₁₁-C₁₄ LAS, e.g., C₁₂ LAS, are a specific example of such surfactants. Another exemplary type of anionic surfactant comprises linear or branched ethoxylated alkyl sulfate surfactants. Such materials, also known as alkyl ether sulfates or alkyl polyethoxylate sulfates, are those which correspond to the formula: R′—O—(C₂H₄₀)ₙ—SO₃M wherein R′ is a C₈-C₂₀ alkyl group, n is from about 1 to 20, and M is a salt-forming cation. In a specific embodiment, R′ is C₁₀-C₁₈ alkyl, n is from about 1 to 15, and M is sodium, potassium, ammonium, alkylammonium, or alkanolammonium. In more specific embodiments, R′ is a C₁₂-C₁₆, n is from about 1 to 6 and M is sodium. The alkyl ether sulfates will generally be used in the form of mixtures comprising varying R′ chain lengths and varying degrees of ethoxylation. Frequently such mixtures will inevitably also contain some non-ethoxylated alkyl sulfate materials, i.e., surfactants of the above ethoxylated alkyl sulfate formula wherein n=0. Non-ethoxylated alkyl sulfates may also be added separately to the compositions of this invention and used as or in any anionic surfactant component which may be present. Specific examples of non-alkoyxylated, e.g., non-ethoxylated, alkyl ether sulfate surfactants are those produced by the sulfation of higher C₈-C₂₀ fatty alcohols. Conventional primary alkyl sulfate surfactants have the general formula: R″OSO₃⁻M⁺ wherein R″ is typically a C₈-C₂₀ alkyl group, which may be straight chain or branched chain, and M is a water-solubilizing cation. In specific embodiments, R″ is a C₁₀-C₁₅ alkyl group, and M is alkali metal, more specifically R″ is C₁₂-C₁₄ alkyl and M is sodium. Specific, non-limiting examples of anionic surfactants useful herein include: a) C₁₁-C₁₈ alkyl benzene sulfonates (LAS); b) C₁₀-C₂₀ primary, branched-chain and random alkyl sulfates (AS); c) C₁₀-C₁₈ secondary (2,3)-alkyl sulfates having following formulae:

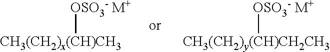

wherein M is hydrogen or a cation which provides charge neutrality, and all M units, whether associated with a surfactant or adjunct ingredient, can either be a hydrogen atom or a cation depending upon the form isolated by the artisan or the relative pH of the system wherein the compound is used, with non-limiting examples of preferred cations including sodium, potassium, ammonium, and mixtures thereof, and x is an integer of at least about 7, preferably at least about 9, and y is an integer of at least 8, preferably at least about 9; d) C₁₀-C₁₈ alkyl alkoxy sulfates (AE_{z}S) wherein preferably z is from 1-30; e) C₁₀-C₁₈ alkyl alkoxy carboxylates preferably comprising 1-5 ethoxy units; f) mid-chain branched alkyl sulfates as discussed in U.S. Pat. Nos. 6,020,303 and 6,060,443; g) mid-chain branched alkyl alkoxy sulfates as discussed in U.S. Pat. Nos. 6,008,181 and 6,020,303; h) modified alkylbenzene sulfonate (MLAS) as discussed in WO 99/05243, WO 99/05242, WO 99/05244, WO 99/05082, WO 99/05084, WO 99/05241, WO 99/07656, WO 00/23549, and WO 00/23548; i) methyl ester sulfonate (MES); and j) alpha-olefin sulfonate (AOS).

Non-limiting examples of nonionic surfactants include: C₁₂-C₁₈ alkyl ethoxylates, such as, NEODOL® nonionic surfactants from Shell; C₆-C₁₂ alkyl phenol alkoxylates wherein the alkoxylate units are a mixture of ethyleneoxy and propyleneoxy units; C₁₂-C₁₈ alcohol and C₆-C₁₂ alkyl phenol condensates with ethylene oxide/propylene oxide block alkyl polyamine ethoxylates such as PLURONIC® from BASF; C₁₄-C₂₂ mid-chain branched alcohols, BA, as discussed in U.S. Pat. No. 6,150,322; C₁₄-C₂₂ mid-chain branched alkyl alkoxylates, BAEₓ, wherein x is from 1-30, as discussed in U.S. Pat. No. 6,153,577, U.S. Pat. No. 6,020,303 and U.S. Pat. No. 6,093,856; alkylpolysaccharides as discussed in U.S. Pat. No. 4,565,647 Llenado, issued Jan. 26, 1986; specifically alkylpolyglycosides as discussed in U.S. Pat. No. 4,483,780 and U.S. Pat. No. 4,483,779; polyhydroxy detergent acid amides as discussed in U.S. Pat. No. 5,332,528; and ether capped poly(oxyalkylated) alcohol surfactants as discussed in U.S. Pat. No. 6,482,994 and WO 01/42408.

Non-limiting examples of semi-polar nonionic surfactants include: water-soluble amine oxides containing one alkyl moiety of from about 10 to about 18 carbon atoms and 2 moieties selected from the group consisting of alkyl moieties and hydroxyalkyl moieties containing from about 1 to about 3 carbon atoms; water-soluble phosphine oxides containing one alkyl moiety of from about 10 to about 18 carbon atoms and 2 moieties selected from the group consisting of alkyl moieties and hydroxyalkyl moieties containing from about 1 to about 3 carbon atoms; and water-soluble sulfoxides containing one alkyl moiety of from about 10 to about 18 carbon atoms and a moiety selected from the group consisting of alkyl moieties and hydroxyalkyl moieties of from about 1 to about 3 carbon atoms. See WO 01/32816, U.S. Pat. No. 4,681,704, and U.S. Pat. No. 4,133,779.

Non-limiting examples of cationic surfactants include: the quaternary ammonium surfactants, which can have up to 26 carbon atoms include: alkoxylate quaternary ammonium (AQA) surfactants as discussed in U.S. Pat. No. 6,136,769; dimethyl hydroxyethyl quaternary ammonium as discussed in 6,004,922; dimethyl hydroxyethyl lauryl ammonium chloride; polyamine cationic surfactants as discussed in WO 98/35002, WO 98/35003, WO 98/35004, WO 98/35005, and WO 98/35006; cationic ester surfactants as discussed in U.S. Pat. Nos. 4,228,042, 4,239,660 4,260,529 and U.S. Pat. No. 6,022,844; and amino surfactants as discussed in U.S. Pat. No. 6,221,825 and WO 00/47708, specifically amido propyldimethyl amine (APA).

Non-limiting examples of zwitterionic or ampholytic or amphoteric surfactants include: derivatives of secondary and tertiary amines, derivatives of heterocyclic secondary and tertiary amines, or derivatives of quaternary ammonium, quaternary phosphonium or tertiary sulfonium compounds. See U.S. Pat. No. 3,929,678 at column 19, line 38 through column 22, line 48, for examples of zwitterionic surfactants; betaines, including alkyl dimethyl betaine and cocodimethyl amidopropyl betaine, C₈ to C₁₈ (for example from C₁₂ to C₁₈) amine oxides and sulfo and hydroxy betaines, such as N-alkyl-N,N-dimethylammino-1-propane sulfonate where the alkyl group can be C₈ to C₁₈ and in certain embodiments from C₁₀ to C₁₄. Non-limiting examples of ampholytic surfactants include: aliphatic derivatives of secondary or tertiary amines, or aliphatic derivatives of heterocyclic secondary and tertiary amines in which the aliphatic radical can be straight- or branched-chain. One of the aliphatic substituents may contain at least about 8 carbon atoms, for example from about 8 to about 18 carbon atoms, and at least one contains an anionic water-solubilizing group, e.g. carboxy, sulfonate, sulfate. See U.S. Pat. No. 3,929,678 at column 19, lines 18-35, for suitable examples of ampholytic surfactants.

Suitable lightly branched surfactants include lightly branched isoprenoid-derived surfactants represented by the following formula:

G-Y-Z

where G is one or more saturated, acyclic C10-C24 isoprenoid-based hydrophobe(s) and Y and Z are as defined below; such lightly branched isoprenoid-based surfactants are exemplified by formulas xii through xv below:

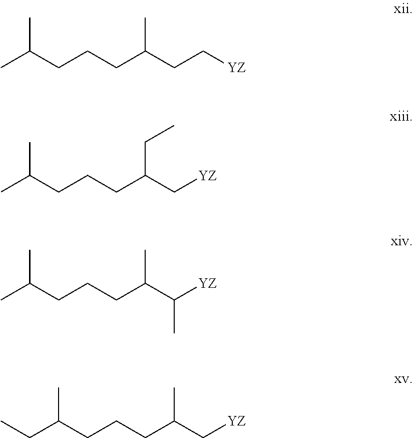

where Y and Z are as defined above.

Additional examples of suitable anionic, zwitterionic, amphoteric, or other surfactants for use in the compositions of the invention are described in U.S. Pat. Nos. 3,929,678; 2,658,072; 2,438,091; 2,528,378; 2,486,921; 2,486,922; 2,396,278; and 3,332,880. Further nonlimiting examples of lightly branched surfactants include those described in U.S. Pat. Nos. 5,870,694, 6,222,077, 5,849,960, and 6,150,322.

In some aspects, the lightly branched surfactant may include a non-isoprenoid-based surfactant, such as those described in US Patent Application Nos. 2011/0171155A1 and 2011/0166370A1, where are hereby incorporated by reference.

Still other lightly branched isoprenoids or isoprenoid derivatives may be found in the book entitled “Comprehensive Natural Products Chemistry: Isoprenoids Including Carotenoids and Steroids (Vol. two)”, Barton and Nakanishi, © 1999, Elsevier Science Ltd and are included in the structures E or G, and are hereby incorporated by reference.

Additional Surfactants

The “highly branched” or “lightly branched” surfactants of the surfactant system of the present invention may include a di-hydrophile substituted isoprenoid-derived surfactant. Highly branched di-hydrophile substituted isoprenoid-derived surfactants comprise a hydrophobe moiety having a main carbon chain with greater than about 2.4 alkyl branches pendant from the main chain. Lightly branched di-hydrophile substituted isoprenoid-derived surfactants comprise a hydrophobe moiety having a main carbon chain with less than about 2.4 alkyl branches pendant from the main chain. The di-hydrophile substituted isoprenoid-derived surfactant may be selected from the following:

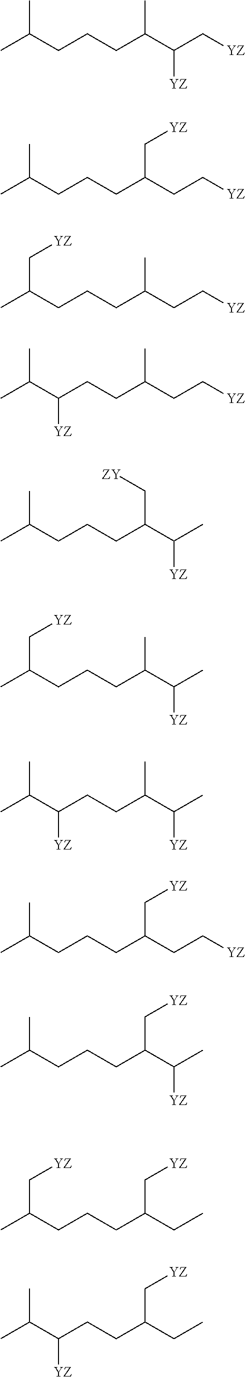

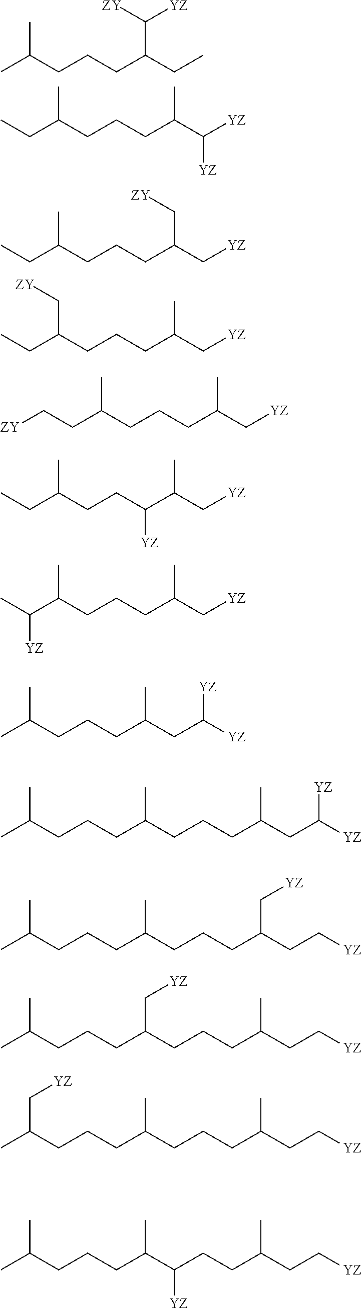

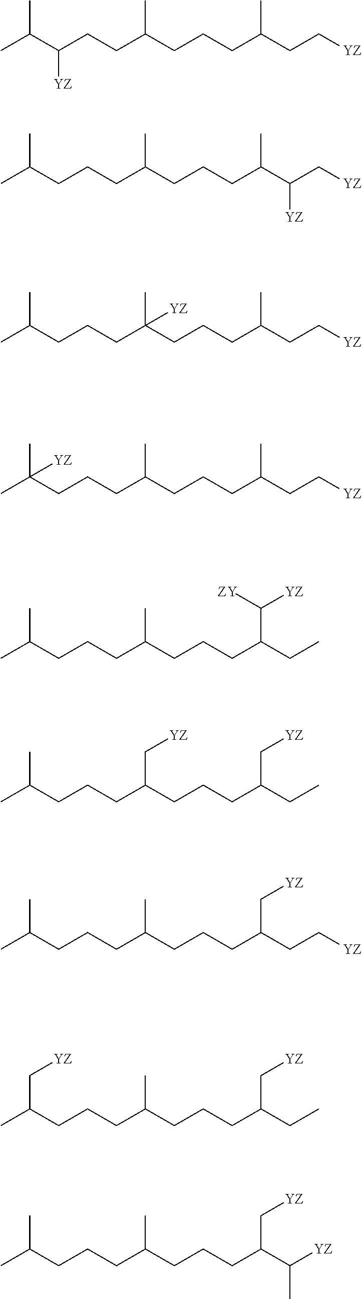

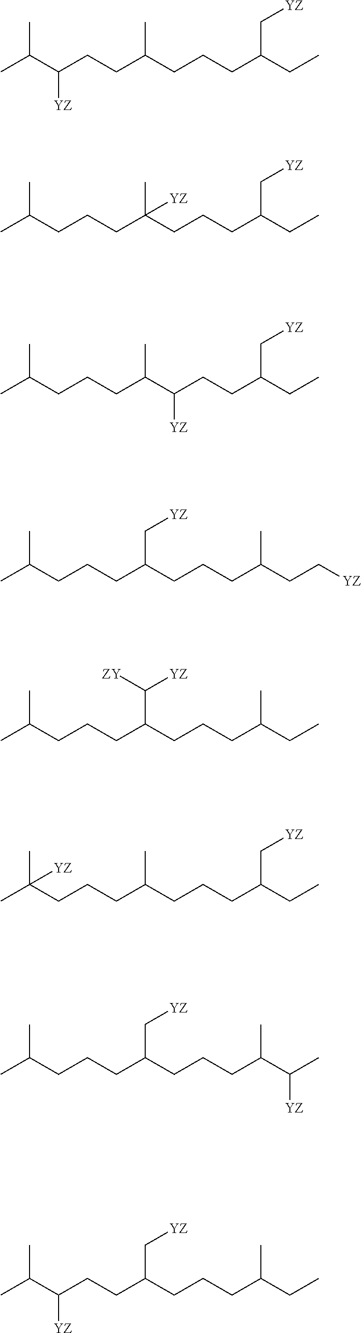

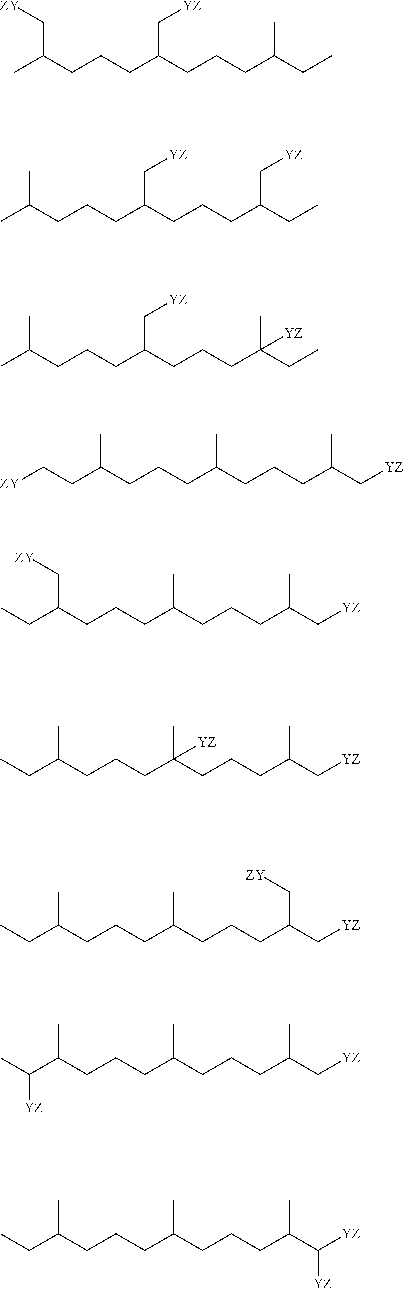

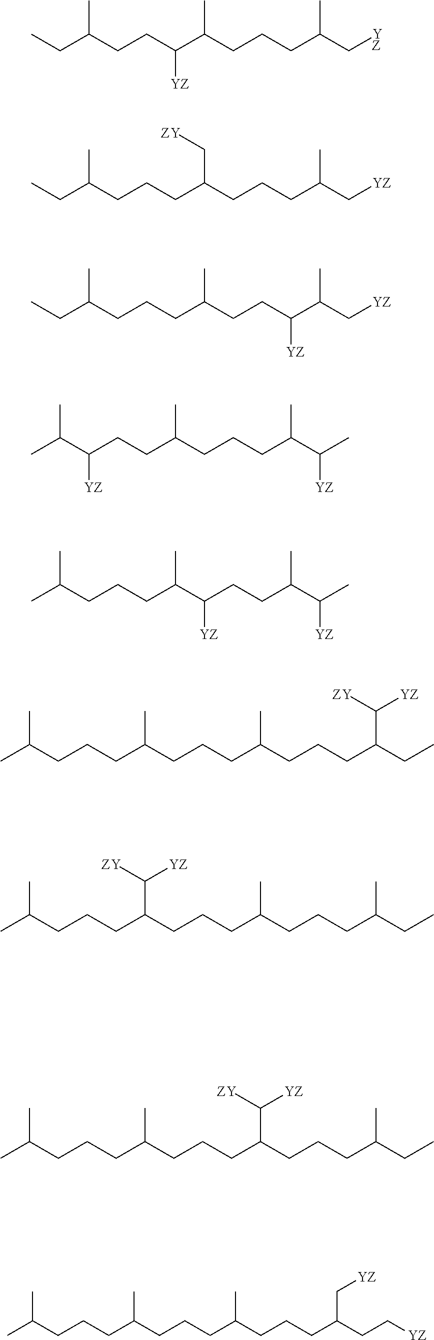

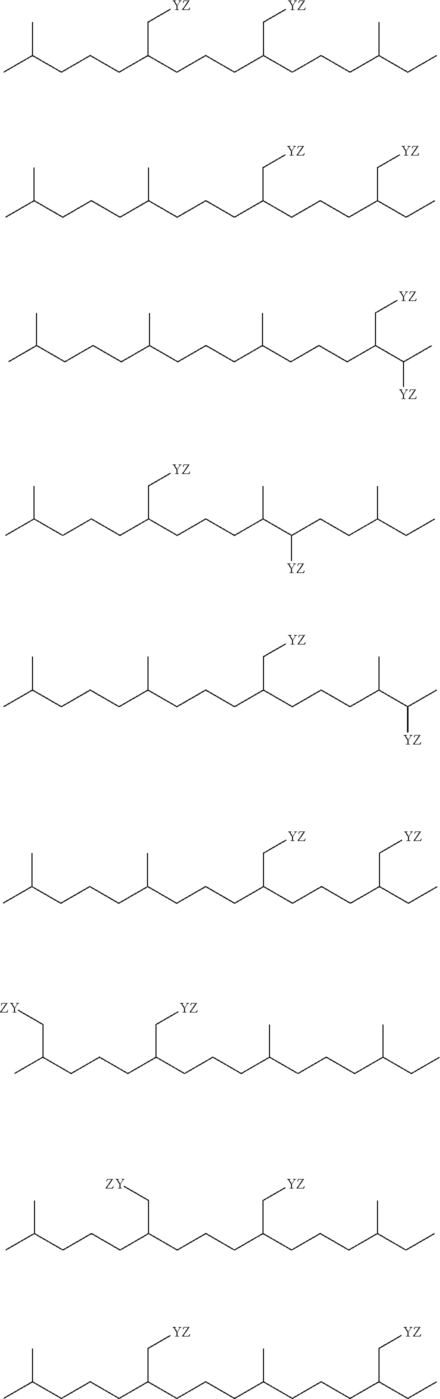

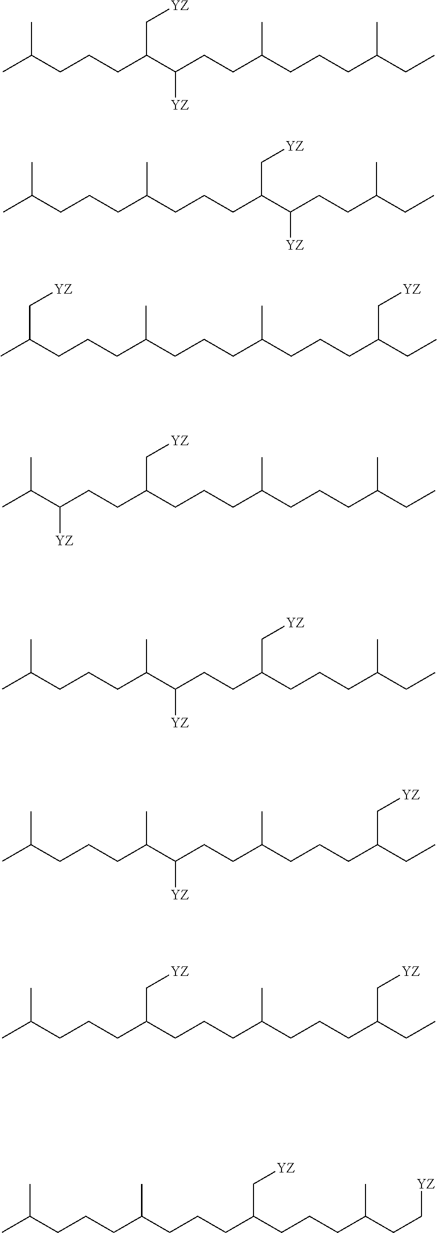

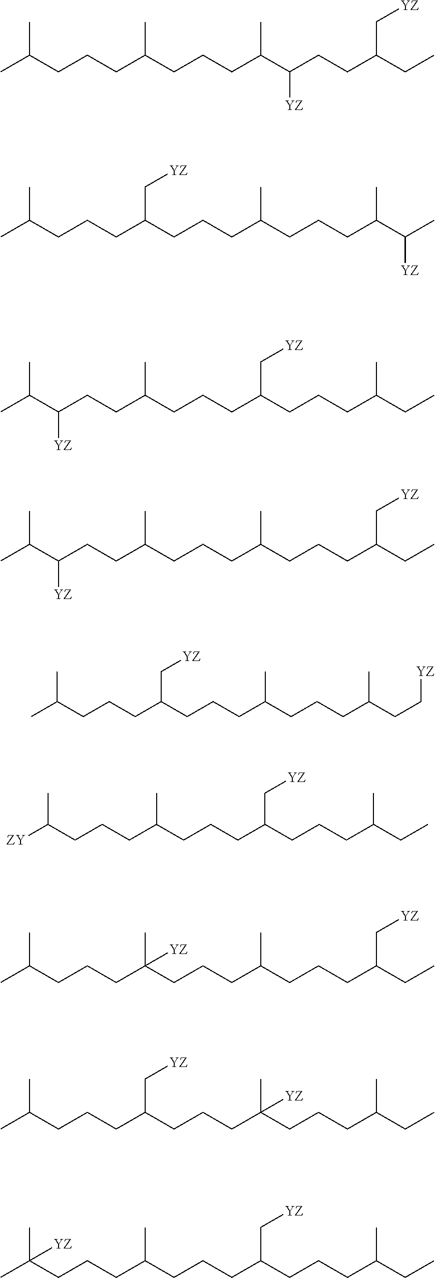

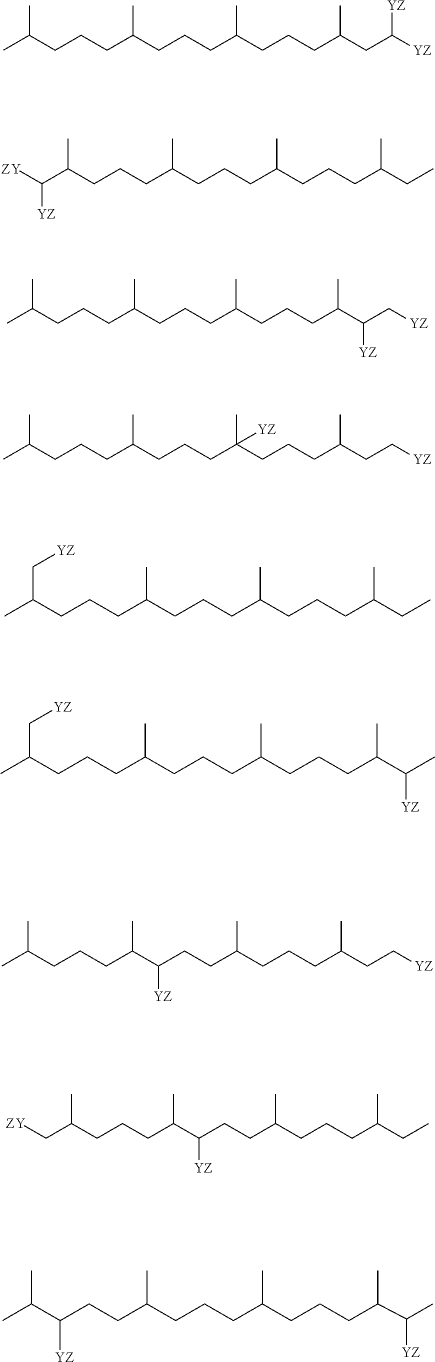

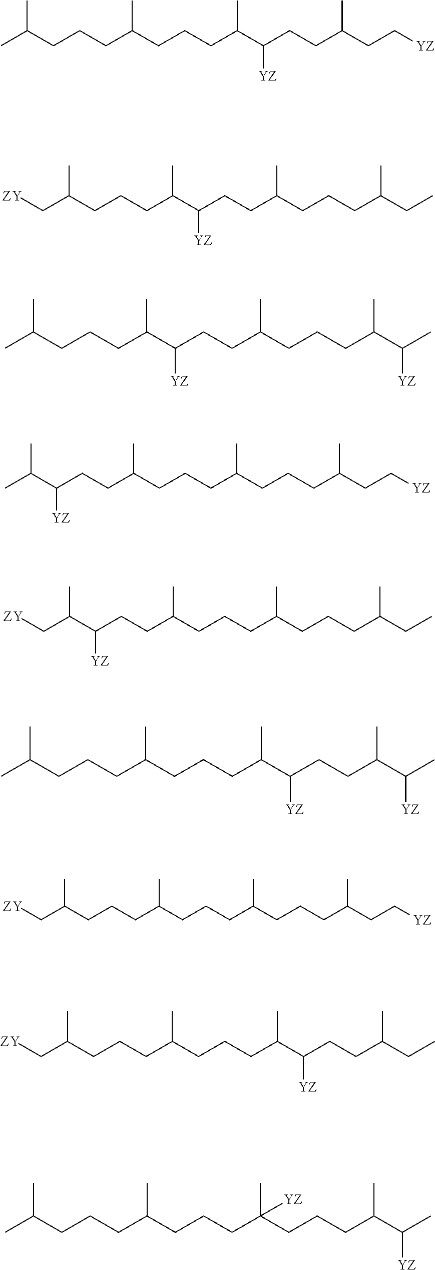

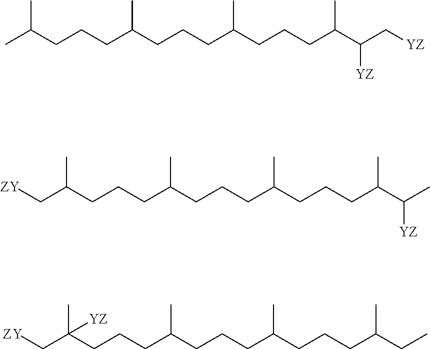

where Y and Z are as defined above. In some aspects, Z is OSO₃⁻, SO₃⁻, hydroxy, O(CH₂CH₂O)ₚH, or O(CH₂CH₂O)ₚSO3⁻; p ranges from about 1 to about 30.

Another aspect of the present invention relates to “highly branched” or “lightly branched” surfactants that have two or more isoprenoid-derived hydrophobes per molecule. The “highly branched” or “lightly branched” surfactants of the surfactant system of the present invention may include a di-isoprenoid-hydrophobe-based surfactant or a multi-isoprenoid-hydrophobe-based-surfactant. These surfactants may be represented by the following formula:

(T-U)ⱼV

where V is a polyhydroxy moiety; a sucrose moiety; a mono-, di-, oligo-, or polysaccharide moiety; a polyglycerol moiety; a polyglycol moiety; a dialkyl ammonium moiety; a dimethylammonium moiety; or a gemini surfactant spacer moiety;
j ranges from 2 to 10, preferably 2, 3, or 4;
U is either absent or is selected from —CO₂—, —CO₂CH₂CH₂—, or a gemini surfactant polar or charged moiety; where if either U or V is a charged moiety, the charged moiety is charge balanced by a suitable counterion;
T is one or more isoprenoid-derived hydrophobe radicals, including but not limited to the following:

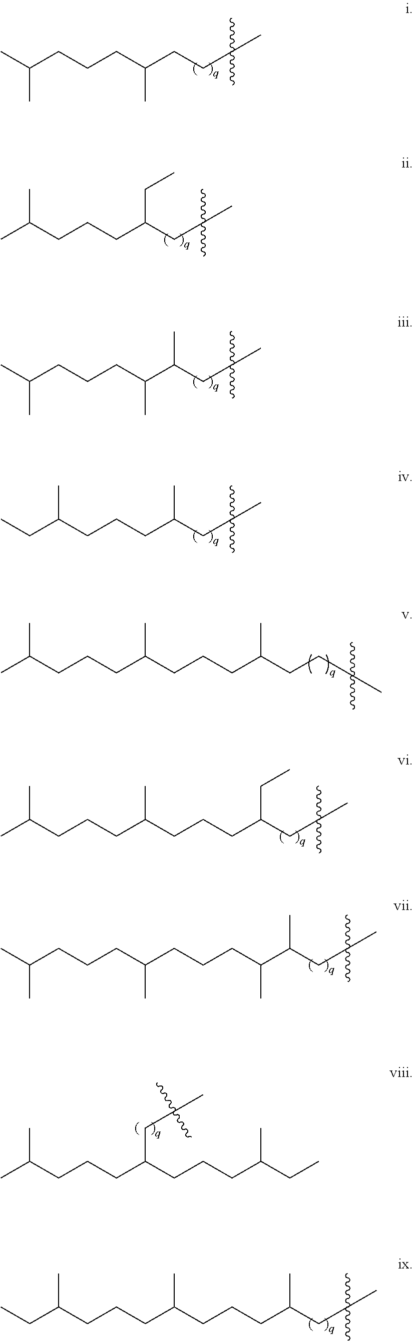

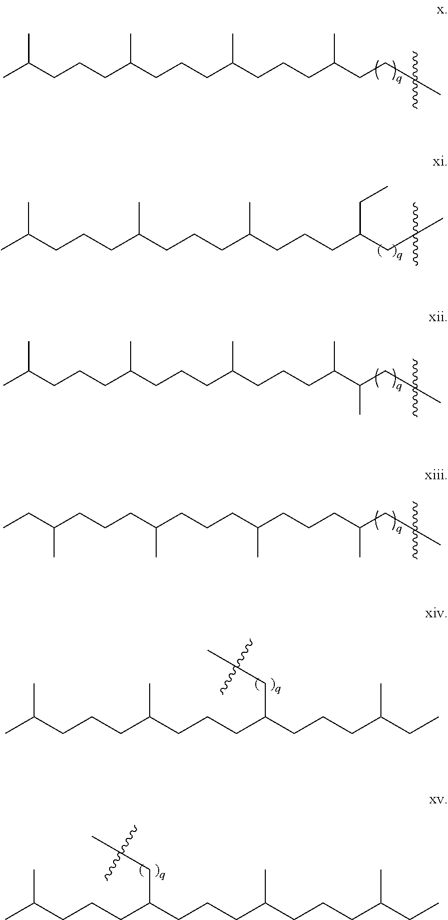

where q is 0-5, preferably 1-2, provided that q may only be zero for structures iii, viii, and xiii above.

In one aspect, (T-U)₂V is a cationic fabric softener active, where U is a spacer moiety or absent, and V is a dialkylammonium moiety, preferably dimethyl ammonium. Non-limiting examples of (T-U)₂V are:

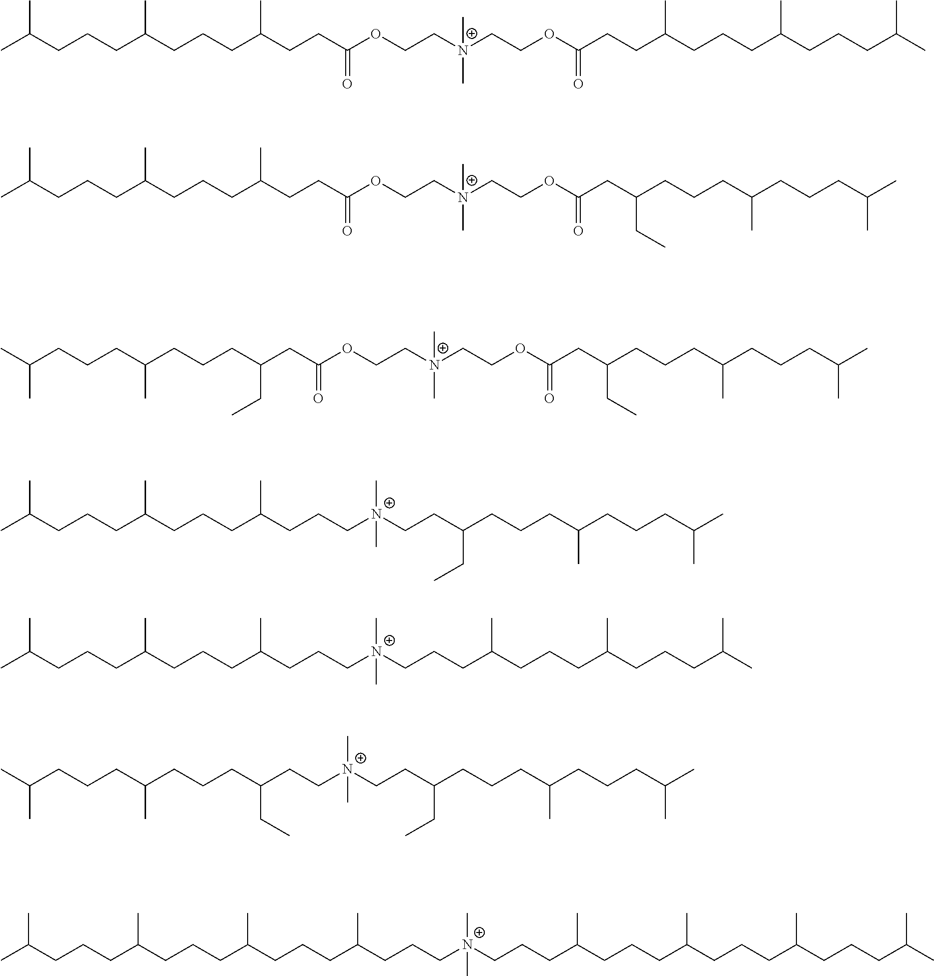

where the cationic moiety is charge balanced by a suitable anion.

Fabric softener compositions containing such di-isoprenoid-hydrophobe cationic surfactants are also included in the scope of the present invention.

In another aspect, (T-U)ⱼV is a di- or poly-T-substituted monosaccharide, disccharide (e.g., sucrose), or oligosaccharide moiety.

In another aspect, (T-U)ⱼV is a gemini surfactant where U is a charged or polar moiety, j is 2-4, preferably 2, and V is a gemini surfactant spacer moiety. As is well known in the art, Gemini surfactants typically (though not always) comprise two hydrophobes separated by a “spacer” moiety and two or more polar headgroups; hence according to the present invention, the T-substituted Gemini surfactants are of the structure:

T-(polar or charged headgroup)-spacer-(polar or charged headgroup)-T.

Suitable structures of said Gemini “polar or charged headgroups” and “spacer” moieties may be found in the surfactant literature, for example, in “Gemini Surfactants: A distinct class of self-assembling Molecules” (S. P Moulik et al., Current Science, vol. 82, No. 9, 10 May 2002) and “Gemini Surfactants” (Surfactant Science Series Vol. 117, Ed. R. Zana, 2003, Taylor & Francis Publishers, Inc), which are hereby incorporated by reference. Additional suitable examples of spacers include —CH2-, —CH2CH2-; —CH2CH2-CH2-; —CH2CH2CH2CH2-; —CH2CH(OH)CH2-; —(CH2)xO(CH2CH2O)yCH2z- wherein x=0-3, y=0-3, z=0-3 and x+y+z>0; —(CH2)xN(CH3)(CH2)y- wherein x=1-3 and y=1-3.

The detergent compositions according to the present invention may further comprise additional surfactants, herein referred to as co-surfactants, which are included at lower levels. Typically, fully-formulated cleaning compositions will contain a mixture of surfactant types in order to obtain broad-scale cleaning performance over a variety of soils and stains and under a variety of usage conditions. A wide range of these co-surfactants can be used in the detergent compositions of the present invention. A typical listing of anionic, nonionic, ampholytic and zwitterionic classes, and species of these co-surfactants, is given herein above, or may also be found in U.S. Pat. No. 3,664,961. The selection of co-surfactant may be dependent upon the desired benefit. The surfactant system may comprise from 0% to about 10%, or from about 0.1% to about 5%, or from about 1% to about 4% by weight of the composition of co-surfactant(s). In some aspects, the co-surfactant comprises a linear or lightly branched surfactant.

### Adjunct Cleaning Additives

The detergent compositions of the invention may also contain adjunct cleaning additives. The adjunct cleaning additives may be selected from builders, structurants or thickeners, clay soil removal/anti-redeposition agents, polymeric soil release agents, polymeric dispersing agents, polymeric grease cleaning agents, enzymes, enzyme stabilizing systems, bleaching compounds, bleaching agents, bleach activators, bleach catalysts, brighteners, dyes, fabric hueing agents, dye transfer inhibiting agents, chelating agents, suds suppressors, fabric softeners, perfumes, or mixtures thereof. This listing of such ingredients is exemplary only, and not by way of limitation of the types of ingredients which can be used with surfactants systems herein. A detailed description of additional components can be found in U.S. Pat. No. 6,020,303.

### Builders

The detergent compositions of the present invention may optionally comprise a builder. Built detergents typically comprise at least about 1 wt % builder, based on the total weight of the detergent. Liquid formulations typically comprise up to about 10 wt %, more typically up to 8 wt % of builder to the total weight of the detergent. Granular formulations typically comprise up to about 30%, more typically from up to 5% builder by weight of the detergent composition.

Detergent builders, when uses are selected from aluminosilicates and silicates to assist in controlling mineral, especially calcium and/or magnesium hardness in wash water or to assist in the removal of particulate soils from surfaces. Suitable builders can be selected from the group consisting of phosphates and polyphosphates, especially the sodium salts; carbonates, bicarbonates, sesquicarbonates and carbonate minerals other than sodium carbonate or sesquicarbonate; organic mono-, di-, tri-, and tetracarboxylates especially water-soluble nonsurfactant carboxylates in acid, sodium, potassium or alkanolammonium salt form, as well as oligomeric or water-soluble low molecular weight polymer carboxylates including aliphatic and aromatic types; and phytic acid. These may be complemented by borates, e.g., for pH-buffering purposes, or by sulfates, especially sodium sulfate and any other fillers or carriers which may be important to the engineering of stable surfactant and/or builder-containing detergent compositions. Other detergent builders can be selected from the polycarboxylate builders, for example, copolymers of acrylic acid, copolymers of acrylic acid and maleic acid, and copolymers of acrylic acid and/or maleic acid and other suitable ethylenic monomers with various types of additional functionalities. Also suitable for use as builders herein are synthesized crystalline ion exchange materials or hydrates thereof having chain structure and a composition represented by the following general Formula I an anhydride form: x(M₂O)·ySiO₂·M′O wherein M is Na and/or K, M′ is Ca and/or Mg; y/x is 0.5 to 2.0 and z/x is 0.005 to 1.0 as taught in U.S. Pat. No. 5,427,711.

However, it has also been found that the isoprenoid-based A and B surfactants are particularly suited to performing well in un-built conditions. Therefore, lower levels of builders, including especially detergents having less than 1% by weight, and in particular builders that are essentially free of builders are of special relevance to the present invention. By “essentially free” it is meant that no builders are intentionally added to the desired detergent composition.

### Structurant/Thickeners

Structured liquids can either be internally structured, whereby the structure is formed by primary ingredients (e.g. surfactant material) and/or externally structured by providing a three dimensional matrix structure using secondary ingredients (e.g. polymers, clay and/or silicate material). The composition may comprise a structurant, preferably from 0.01 wt % to 5 wt %, from 0.1 wt % to 2.0 wt % structurant. The structurant is typically selected from the group consisting of diglycerides and triglycerides, ethylene glycol distearate, microcrystalline cellulose, cellulose-based materials, microfiber cellulose, biopolymers, xanthan gum, gellan gum, and mixtures thereof. A suitable structurant includes hydrogenated castor oil, and non-ethoxylated derivatives thereof. A suitable structurant is disclosed in U.S. Pat. No. 6,855,680. Such structurants have a thread-like structuring system having a range of aspect ratios. Other suitable structurants and the processes for making them are described in WO2010/034736.

### Clay Soil Removal/Anti-Redeposition Agents

The compositions of the present invention can also optionally contain water-soluble ethoxylated amines having clay soil removal and antiredeposition properties. Granular detergent compositions which contain these compounds typically contain from about 0.01% to about 10.0% by weight of the water-soluble ethoxylates amines; liquid detergent compositions typically contain about 0.01% to about 5% by weight.

Exemplary clay soil removal and antiredeposition agents are described in U.S. Pat. Nos. 4,597,898; 548,744; 4,891,160; European Patent Application Nos. 111,965; 111,984; 112,592; and WO 95/32272.

### Polymeric Soil Release Agent

Known polymeric soil release agents, hereinafter “SRA” or “SRA's”, can optionally be employed in the present detergent compositions. If utilized, SRA's will generally comprise from 0.01% to 10.0%, typically from 0.1% to 5%, preferably from 0.2% to 3.0% by weight, of the composition.

Preferred SRA's typically have hydrophilic segments to hydrophilize the surface of hydrophobic fibers such as polyester and nylon, and hydrophobic segments to deposit upon hydrophobic fibers and remain adhered thereto through completion of washing and rinsing cycles thereby serving as an anchor for the hydrophilic segments. This can enable stains occurring subsequent to treatment with SRA to be more easily cleaned in later washing procedures.

SRA's can include, for example, a variety of charged, e.g., anionic or even cationic (see U.S. Pat. No. 4,956,447), as well as noncharged monomer units and structures may be linear, branched or even star-shaped. They may include capping moieties which are especially effective in controlling molecular weight or altering the physical or surface-active properties. Structures and charge distributions may be tailored for application to different fiber or textile types and for varied detergent or detergent additive products. Examples of SRAs are described in U.S. Pat. Nos. 4,968,451; 4,711,730; 4,721,580; 4,702,857; 4,877,896; 3,959,230; 3,893,929; 4,000,093; 5,415,807; 4,201,824; 4,240,918; 4,525,524; 4,201,824; 4,579,681; and 4,787,989; European Patent Application 0 219 048; 279,134 A; 457,205 A; and DE 2,335,044.

### Polymeric Dispersing Agents

Polymeric dispersing agents can advantageously be utilized at levels from about 0.1% to about 7%, by weight, in the compositions herein, especially in the presence of zeolite and/or layered silicate builders. Suitable polymeric dispersing agents include polymeric polycarboxylates and polyethylene glycols, although others known in the art can also be used. For example, a wide variety of modified or unmodified polyacrylates, polyacrylate/mealeates, or polyacrylate/methacrylates are highly useful. It is believed, though it is not intended to be limited by theory, that polymeric dispersing agents enhance overall detergent builder performance, when used in combination with other builders (including lower molecular weight polycarboxylates) by crystal growth inhibition, particulate soil release peptization, and anti-redeposition. Examples of polymeric dispersing agents are found in U.S. Pat. No. 3,308,067, European Patent Application No. 66915, EP 193,360, and EP 193,360.

### Alkoxylated Polyamine Polymers

Soil suspension, grease cleaning, and particulate cleaning polymers may include the alkoxylated polyamines. Such materials include but are not limited to ethoxylated polyethyleneimine, ethoxylated hexamethylene diamine, and sulfated versions thereof. Polypropoxylated derivatives are also included. A wide variety of amines and polyaklyeneimines can be alkoxylated to various degrees, and optionally further modified to provide the abovementioned benefits. A useful example is 600 g/mol polyethyleneimine core ethoxylated to 20 EO groups per NH and is available from BASF.

### Polymeric Grease Cleaning Polymers

Alkoxylated polycarboxylates such as those prepared from polyacrylates are useful herein to provide additional grease removal performance. Such materials are described in WO 91/08281 and PCT 90/01815. Chemically, these materials comprise polyacrylates having one ethoxy side-chain per every 7-8 acrylate units. The side-chains are of the formula —(CH₂CH₂O)ₘ (CH₂)ₙCH₃ wherein m is 2-3 and n is 6-12. The side-chains are ester-linked to the polyacrylate “backbone” to provide a “comb” polymer type structure. The molecular weight can vary, but is typically in the range of about 2000 to about 50,000. Such alkoxylated polycarboxylates can comprise from about 0.05% to about 10%, by weight, of the compositions herein.

The isoprenoid-derived surfactants of the present invention, and their mixtures with other co-surfactants and other adjunct ingredients, are particularly suited to be used with an amphiphilic graft co-polymer, preferably the amphiphilic graft co-polymer comprises (i) polyethyelene glycol backbone; and (ii) and at least one pendant moiety selected from polyvinyl acetate, polyvinyl alcohol and mixtures thereof. A preferred amphiphilic graft co-polymer is Sokalan HP22, supplied from BASF.

### Enzymes

Enzymes, including proteases, amylases, other carbohydrases, lipases, oxidases, and cellulases may be used as adjunct ingredients. Enzymes are included in the present cleaning compositions for a variety of purposes, including removal of protein-based, carbohydrate-based, or triglyceride-based stains from substrates, for the prevention of refugee dye transfer in fabric laundering, and for fabric restoration. Suitable enzymes include proteases, amylases, lipases, cellulases, peroxidases, and mixtures thereof of any suitable origin, such as vegetable, animal, bacterial, fungal and yeast origin. Preferred selections are influenced by factors such as pH-activity and/or stability optima, thermostability, and stability to active detergents, builders and the like. In this respect bacterial or fungal enzymes are preferred, such as bacterial amylases and proteases, and fungal cellulases.

Enzymes are normally incorporated into detergent or detergent additive compositions at levels sufficient to provide a “cleaning-effective amount”. The term “cleaning effective amount” refers to any amount capable of producing a cleaning, stain removal, soil removal, whitening, deodorizing, or freshness improving effect on substrates such as fabrics, dishware and the like. In practical terms for current commercial preparations, typical amounts are up to about 5 mg by weight, more typically 0.01 mg to 3 mg, of active enzyme per gram of the household cleaning composition. Stated otherwise, the compositions herein will typically comprise from 0.001% to 5%, preferably 0.01%-1% by weight of a commercial enzyme preparation.

A range of enzyme materials and means for their incorporation into synthetic detergent compositions is also disclosed in WO 9307263 A; WO 9307260 A; WO 8908694 A; U.S. Pat. Nos. 3,553,139; 4,101,457; and U.S. Pat. No. 4,507,219. Enzyme materials useful for liquid detergent formulations, and their incorporation into such formulations, are disclosed in U.S. Pat. No. 4,261,868. Enzymes for use in detergents can be stabilized by various techniques. Enzyme stabilization techniques are disclosed and exemplified in U.S. Pat. Nos. 3,600,319 and 3,519,570; EP 199,405, EP 200,586; and WO 9401532 A.

### Enzyme Stabilizing System

The enzyme-containing compositions herein may optionally also comprise from about 0.001% to about 10%, preferably from about 0.005% to about 8%, most preferably from about 0.01% to about 6%, by weight of an enzyme stabilizing system. The enzyme stabilizing system can be any stabilizing system which is compatible with the detersive enzyme. Such a system may be inherently provided by other formulation actives, or be added separately, e.g., by the formulator or by a manufacturer of detergent-ready enzymes. Such stabilizing systems can, for example, comprise calcium ion, boric acid, propylene glycol, short chain carboxylic acids, boronic acids, and mixtures thereof, and are designed to address different stabilization problems depending on the type and physical form of the detergent composition.

### Bleaching Compounds, Bleaching Agents, Bleach Activators, and Bleach Catalysts

The cleaning compositions herein may further contain bleaching agents or bleaching compositions containing a bleaching agent and one or more bleach activators. Bleaching agents will typically be at levels of from about 1 wt % to about 30 wt %, more typically from about 5 wt % to about 20 wt %, based on the total weight of the composition, especially for fabric laundering. If present, the amount of bleach activators will typically be from about 0.1 wt % to about 60 wt %, more typically from about 0.5 wt % to about 40 wt % of the bleaching composition comprising the bleaching agent-plus-bleach activator.

Examples of bleaching agents include oxygen bleach, perborate bleach, percarboxylic acid bleach and salts thereof, peroxygen bleach, persulfate bleach, percarbonate bleach, and mixtures thereof. Examples of bleaching agents are disclosed in U.S. Pat. No. 4,483,781, U.S. patent application Ser. No. 740,446, European Patent Application 0,133,354, U.S. Pat. No. 4,412,934, and U.S. Pat. No. 4,634,551.

Examples of bleach activators (e.g., acyl lactam activators) are disclosed in U.S. Pat. Nos. 4,915,854; 4,412,934; 4,634,551; 4,634,551; and 4,966,723.

Preferably, a laundry detergent composition comprises a transition metal catalyst. Preferably, the transition metal catalyst may be encapsulated. The transition metal bleach catalyst typically comprises a transition metal ion, preferably selected from transition metal selected from the group consisting of Mn(II), Mn(III), Mn(IV), Mn(V), Fe(II), Fe(III), Fe(IV), Co(I), Co(II), Co(III), Ni(I), Ni(II), Ni(III), Cu(I), Cu(II), Cu(III), Cr(II), Cr(III), Cr(IV), Cr(V), Cr(VI), V(III), V(IV), V(V), Mo(IV), Mo(V), Mo(VI), W(IV), W(V), W(VI), Pd(II), Ru(II), Ru(III), and Ru(IV), more preferably Mn(II), Mn(III), Mn(IV), Fe(II), Fe(III), Cr(II), Cr(III), Cr(IV), Cr(V), and Cr(VI). The transition metal bleach catalyst typically comprises a ligand, preferably a macropolycyclic ligand, more preferably a cross-bridged macropolycyclic ligand. The transition metal ion is preferably coordinated with the ligand. Preferably, the ligand comprises at least four donor atoms, at least two of which are bridgehead donor atoms. Suitable transition metal bleach catalysts are described in U.S. Pat. No. 5,580,485, U.S. Pat. No. 4,430,243; U.S. Pat. No. 4,728,455; U.S. Pat. No. 5,246,621; U.S. Pat. No. 5,244,594; U.S. Pat. No. 5,284,944; U.S. Pat. No. 5,194,416; U.S. Pat. No. 5,246,612; U.S. Pat. No. 5,256,779; U.S. Pat. No. 5,280,117; U.S. Pat. No. 5,274,147; U.S. Pat. No. 5,153,161; U.S. Pat. No. 5,227,084; U.S. Pat. No. 5,114,606; U.S. Pat. No. 5,114,611, EP 549,271 A1; EP 544,490 A1; EP 549,272 A1; and EP 544,440 A2. A suitable transition metal bleach catalyst is a manganese-based catalyst, for example disclosed in U.S. Pat. No. 5,576,282. Suitable cobalt bleach catalysts are described, for example, in U.S. Pat. No. 5,597,936 and U.S. Pat. No. 5,595,967. Such cobalt catalysts are readily prepared by known procedures, such as taught for example in U.S. Pat. No. 5,597,936, and U.S. Pat. No. 5,595,967. A suitable transition metal bleach catalyst is a transition metal complex of ligand such as bispidones described in WO 05/042532 A1.

Bleaching agents other than oxygen bleaching agents are also known in the art and can be utilized herein (e.g., photoactivated bleaching agents such as the sulfonated zinc and/or aluminum phthalocyanines (U.S. Pat. No. 4,033,718, incorporated herein by reference), or pre-formed organic peracids, such as peroxycarboxylic acid or salt thereof, or a peroxysulphonic acid or salt thereof. A suitable organic peracid is phthaloylimidoperoxycaproic acid. If used, household cleaning compositions will typically contain from about 0.025% to about 1.25%, by weight, of such bleaches, especially sulfonate zinc phthalocyanine.

### Brighteners

Any optical brighteners or other brightening or whitening agents known in the art can be incorporated at levels typically from about 0.01% to about 1.2%, by weight, into the cleaning compositions herein. Commercial optical brighteners which may be useful in the present invention can be classified into subgroups, which include, but are not necessarily limited to, derivatives of stilbene, pyrazoline, coumarin, carboxylic acid, methinecyanines, dibenzothiophene-5,5-dioxide, azoles, 5- and 6-membered-ring heterocycles, and other miscellaneous agents. Examples of such brighteners are disclosed in “The Production and Application of Fluorescent Brightening Agents”, M. Zahradnik, Published by John Wiley & Sons, New York (1982). Specific nonlimiting examples of optical brighteners which are useful in the present compositions are those identified in U.S. Pat. No. 4,790,856 and U.S. Pat. No. 3,646,015.

### Fabric Hueing Agents

The composition may comprise a fabric hueing agent (sometimes referred to as shading, bluing or whitening agents). Typically the hueing agent provides a blue or violet shade to fabric. Hueing agents can be used either alone or in combination to create a specific shade of hueing and/or to shade different fabric types. This may be provided for example by mixing a red and green-blue dye to yield a blue or violet shade. Hueing agents may be selected from any known chemical class of dye, including but not limited to acridine, anthraquinone (including polycyclic quinones), azine, azo (e.g., monoazo, disazo, trisazo, tetrakisazo, polyazo), including premetallized azo, benzodifurane and benzodifuranone, carotenoid, coumarin, cyanine, diazahemicyanine, diphenylmethane, formazan, hemicyanine, indigoids, methane, naphthalimides, naphthoquinone, nitro and nitroso, oxazine, phthalocyanine, pyrazoles, stilbene, styryl, triarylmethane, triphenylmethane, xanthenes and mixtures thereof.

Suitable fabric hueing agents include dyes, dye-clay conjugates, and organic and inorganic pigments. Suitable dyes include small molecule dyes and polymeric dyes. Suitable small molecule dyes include small molecule dyes selected from the group consisting of dyes falling into the Colour Index (C.I.) classifications of Direct, Basic, Reactive or hydrolysed Reactive, Solvent or Disperse dyes for example that are classified as Blue, Violet, Red, Green or Black, and provide the desired shade either alone or in combination. In another aspect, suitable small molecule dyes include small molecule dyes selected from the group consisting of Colour Index (Society of Dyers and Colourists, Bradford, UK) numbers Direct Violet dyes such as 9, 35, 48, 51, 66, and 99, Direct Blue dyes such as 1, 71, 80 and 279, Acid Red dyes such as 17, 73, 52, 88 and 150, Acid Violet dyes such as 15, 17, 24, 43, 49 and 50, Acid Blue dyes such as 15, 17, 25, 29, 40, 45, 75, 80, 83, 90 and 113, Acid Black dyes such as 1, Basic Violet dyes such as 1, 3, 4, 10 and 35, Basic Blue dyes such as 3, 16, 22, 47, 66, 75 and 159, Disperse or Solvent dyes such as those described in EP1794275 or EP1794276, or dyes as disclosed in U.S. Pat. No. 7,208,459 B2, and mixtures thereof. In another aspect, suitable small molecule dyes include small molecule dyes selected from the group consisting of C. I. numbers Acid Violet 17, Direct Blue 71, Direct Violet 51, Direct Blue 1, Acid Red 88, Acid Red 150, Acid Blue 29, Acid Blue 113 or mixtures thereof.

Suitable polymeric dyes include polymeric dyes selected from the group consisting of polymers containing covalently bound (sometimes referred to as conjugated) chromogens, (dye-polymer conjugates), for example polymers with chromogens co-polymerized into the backbone of the polymer and mixtures thereof. Polymeric dyes include those described in WO2011/98355, WO2011/47987, US2012/090102, WO2010/145887, WO2006/055787 and WO2010/142503.

In another aspect, suitable polymeric dyes include polymeric dyes selected from the group consisting of fabric-substantive colorants sold under the name of Liquitint® (Milliken, Spartanburg, S.C., USA), dye-polymer conjugates formed from at least one reactive dye and a polymer selected from the group consisting of polymers comprising a moiety selected from the group consisting of a hydroxyl moiety, a primary amine moiety, a secondary amine moiety, a thiol moiety and mixtures thereof. In still another aspect, suitable polymeric dyes include polymeric dyes selected from the group consisting of Liquitint® Violet CT, carboxymethyl cellulose (CMC) covalently bound to a reactive blue, reactive violet or reactive red dye such as CMC conjugated with C.I. Reactive Blue 19, sold by Megazyme, Wicklow, Ireland under the product name AZO-CM-CELLULOSE, product code S-ACMC, alkoxylated triphenyl-methane polymeric colourants, alkoxylated thiophene polymeric colourants, and mixtures thereof.

Preferred hueing dyes include the whitening agents found in WO 08/87497 A1, WO2011/011799 and WO2012/054835. Preferred hueing agents for use in the present invention may be the preferred dyes disclosed in these references, including those selected from Examples 1-42 in Table 5 of WO2011/011799. Other preferred dyes are disclosed in U.S. Pat. No. 8,138,222. Other preferred dyes are disclosed in WO2009/069077.

Suitable dye clay conjugates include dye clay conjugates selected from the group comprising at least one cationic/basic dye and a smectite clay, and mixtures thereof. In another aspect, suitable dye clay conjugates include dye clay conjugates selected from the group consisting of one cationic/basic dye selected from the group consisting of C.I. Basic Yellow 1 through 108, C.I. Basic Orange 1 through 69, C.I. Basic Red 1 through 118, C.I. Basic Violet 1 through 51, C.I. Basic Blue 1 through 164, C.I. Basic Green 1 through 14, C.I. Basic Brown 1 through 23, CI Basic Black 1 through 11, and a clay selected from the group consisting of Montmorillonite clay, Hectorite clay, Saponite clay and mixtures thereof. In still another aspect, suitable dye clay conjugates include dye clay conjugates selected from the group consisting of: Montmorillonite Basic Blue B7 C.I. 42595 conjugate, Montmorillonite Basic Blue B9 C.I. 52015 conjugate, Montmorillonite Basic Violet V3 C.I. 42555 conjugate, Montmorillonite Basic Green G1 C.I. 42040 conjugate, Montmorillonite Basic Red R1 C.I. 45160 conjugate, Montmorillonite C.I. Basic Black 2 conjugate, Hectorite Basic Blue B7 C.I. 42595 conjugate, Hectorite Basic Blue B9 C.I. 52015 conjugate, Hectorite Basic Violet V3 C.I. 42555 conjugate, Hectorite Basic Green G1 C.I. 42040 conjugate, Hectorite Basic Red R1 C.I. 45160 conjugate, Hectorite C.I. Basic Black 2 conjugate, Saponite Basic Blue B7 C.I. 42595 conjugate, Saponite Basic Blue B9 C.I. 52015 conjugate, Saponite Basic Violet V3 C.I. 42555 conjugate, Saponite Basic Green G1 C.I. 42040 conjugate, Saponite Basic Red R1 C.I. 45160 conjugate, Saponite C.I. Basic Black 2 conjugate and mixtures thereof.

Suitable pigments include pigments selected from the group consisting of flavanthrone, indanthrone, chlorinated indanthrone containing from 1 to 4 chlorine atoms, pyranthrone, dichloropyranthrone, monobromodichloropyranthrone, dibromodichloropyranthrone, tetrabromopyranthrone, perylene-3,4,9,10-tetracarboxylic acid diimide, wherein the imide groups may be unsubstituted or substituted by C1-C3-alkyl or a phenyl or heterocyclic radical, and wherein the phenyl and heterocyclic radicals may additionally carry substituents which do not confer solubility in water, anthrapyrimidinecarboxylic acid amides, violanthrone, isoviolanthrone, dioxazine pigments, copper phthalocyanine which may contain up to 2 chlorine atoms per molecule, polychloro-copper phthalocyanine or polybromochloro-copper phthalocyanine containing up to 14 bromine atoms per molecule and mixtures thereof.

In another aspect, suitable pigments include pigments selected from the group consisting of Ultramarine Blue (C.I. Pigment Blue 29), Ultramarine Violet (C.I. Pigment Violet 15) and mixtures thereof.

The aforementioned fabric hueing agents can be used in combination (any mixture of fabric hueing agents can be used).

### Chelating Agents

The detergent compositions herein may also optionally contain one or more iron and/or manganese and/or other metal ion chelating agents. Such chelating agents can be selected from the group consisting of amino carboxylates, amino phosphonates, polyfunctionally-substituted aromatic chelating agents and mixtures therein. If utilized, these chelating agents will generally comprise from about 0.1% to about 15% by weight of the detergent compositions herein. More preferably, if utilized, the chelating agents will comprise from about 0.1% to about 3.0% by weight of such compositions.

The chelant or combination of chelants may be chosen by one skilled in the art to provide for heavy metal (e.g. Fe) sequestration without negatively impacting enzyme stability through the excessive binding of calcium ions. Non-limiting examples of chelants of use in the present invention are found in U.S. Pat. Nos. 7,445,644, 7,585,376 and 2009/0176684A1.

Useful chelants include heavy metal chelating agents, such as diethylenetriaminepentaacetic acid (DTPA) and/or a catechol including, but not limited to, Tiron. In embodiments in which a dual chelant system is used, the chelants may be DTPA and Tiron.

DTPA has the following core molecular structure:

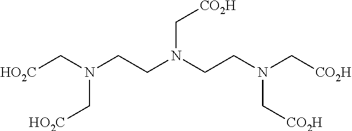 [SMILES: O=C(O)CN(CCN(CC(=O)O)CC(=O)O)CCN(CC(=O)O)CC(=O)O]

Tiron, also known as 1,2-dihydroxybenzene-3,5-disulfonic acid, is one member of the catechol family and has the core molecular structure shown below:

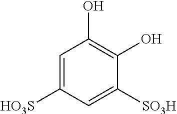 [SMILES: O=S(=O)(O)c1cc(O)c(O)c(S(=O)(=O)O)c1]

Other sulphonated catechols are of use. In addition to the disulfonic acid, the term “tiron” may also include mono- or di-sulfonate salts of the acid, such as, for example, the disodium sulfonate salt, which shares the same core molecular structure with the disulfonic acid.

Other chelating agents suitable for use herein can be selected from the group consisting of aminocarboxylates, aminophosphonates, polyfunctionally-substituted aromatic chelating agents and mixtures thereof. Chelants particularly of use include, but are not limited to: HEDP (hydroxyethanedimethylenephosphonic acid); MGDA (methylglycinediacetic acid); and mixtures thereof.

Without intending to be bound by theory, it is believed that the benefit of these materials is due in part to their exceptional ability to remove heavy metal ions from washing solutions by formation of soluble chelates; other benefits include inorganic film or scale prevention. Other suitable chelating agents for use herein are the commercial DEQUEST series, and chelants from Monsanto, DuPont, and Nalco, Inc.

Aminocarboxylates useful as chelating agents include, but are not limited to, ethylenediaminetetracetates, N-(hydroxyethyl)ethylenediaminetriacetates, nitrilotriacetates, ethylenediamine tetraproprionates, triethylenetetraaminehexacetates, diethylenetriamine-pentaacetates, and ethanoldiglycines, alkali metal, ammonium, and substituted ammonium salts thereof and mixtures thereof. Aminophosphonates are also suitable for use as chelating agents in the compositions of the invention when at least low levels of total phosphorus are permitted in detergent compositions, and include ethylenediaminetetrakis(methylenephosphonates). Preferably, these aminophosphonates do not contain alkyl or alkenyl groups with more than about 6 carbon atoms. Polyfunctionally-substituted aromatic chelating agents are also useful in the compositions herein. See U.S. Pat. No. 3,812,044, issued May 21, 1974, to Connor et al. Preferred compounds of this type in acid form are dihydroxydisulfobenzenes such as 1,2-dihydroxy-3,5-disulfobenzene.

A biodegradable chelator for use herein is ethylenediamine disuccinate (“EDDS”), especially (but not limited to) the [S,S] isomer as described in U.S. Pat. No. 4,704,233. The trisodium salt is preferred though other forms, such as magnesium salts, may also be useful. The chelant system may be present in the detergent compositions of the present invention at from about 0.2% to about 0.7% or from about 0.3% to about 0.6% by weight of the detergent compositions disclosed herein.

### Suds Suppressors

Compounds for reducing or suppressing the formation of suds can be incorporated into the compositions of the present invention. Suds suppression can be of particular importance in the so-called “high concentration cleaning process” as described in U.S. Pat. Nos. 4,489,455 and 4,489,574, and in front-loading-style washing machines.

A wide variety of materials may be used as suds suppressors, and suds suppressors are well known to those skilled in the art. See, for example, Kirk Othmer Encyclopedia of Chemical Technology, Third Edition, Volume 7, pages 430-447 (John Wiley & Sons, Inc., 1979). Examples of suds suppressors include monocarboxylic fatty acid and soluble salts therein, high molecular weight hydrocarbons such as paraffin, fatty acid esters (e.g., fatty acid triglycerides), fatty acid esters of monovalent alcohols, aliphatic C₁₈-C₄₀ ketones (e.g., stearone), N-alkylated amino triazines, waxy hydrocarbons preferably having a melting point below about 100° C., silicone suds suppressors, and secondary alcohols. Suds suppressors are described in U.S. Pat. No. 2,954,347; U.S. Pat. No. 4,265,779; U.S. Pat. No. 4,265,779; U.S. Pat. No. 3,455,839; U.S. Pat. No. 3,933,672; U.S. Pat. No. 4,652,392; U.S. Pat. No. 4,978,471; U.S. Pat. No. 4,983,316; U.S. Pat. No. 5,288,431; U.S. Pat. No. 4,639,489; U.S. Pat. No. 4,749,740; and U.S. Pat. No. 4,798,679; U.S. Pat. No. 4,075,118; European Patent Application No. 89307851.9; EP 150,872; and DOS 2,124,526.

For any detergent compositions to be used in automatic laundry washing machines, suds should not form to the extent that they overflow the washing machine. Suds suppressors, when utilized, are preferably present in a “suds suppressing amount. By “suds suppressing amount” is meant that the formulator of the composition can select an amount of this suds controlling agent that will sufficiently control the suds to result in a low-sudsing laundry detergent for use in automatic laundry washing machines.

The compositions herein will generally comprise from 0% to about 10% of suds suppressor. When utilized as suds suppressors, monocarboxylic fatty acids, and salts therein, will be present typically in amounts up to about 5%, by weight, of the detergent composition. Preferably, from about 0.5% to about 3% of fatty monocarboxylate suds suppressor is utilized. Silicone suds suppressors are typically utilized in amounts up to about 2.0%, by weight, of the detergent composition, although higher amounts may be used. Monostearyl phosphate suds suppressors are generally utilized in amounts ranging from about 0.1% to about 2%, by weight, of the composition. Hydrocarbon suds suppressors are typically utilized in amounts ranging from about 0.01% to about 5.0%, although higher levels can be used. The alcohol suds suppressors are typically used at 0.2%-3% by weight of the finished compositions.

### Fabric Softeners

Various through-the-wash fabric softeners, especially the impalpable smectite clays of U.S. Pat. No. 4,062,647, as well as other softener clays known in the art, can optionally be used typically at levels of from about 0.5% to about 10% by weight in the present compositions to provide fabric softener benefits concurrently with fabric cleaning. Clay softeners can be used in combination with amine and cationic softeners as disclosed, for example, in U.S. Pat. No. 4,375,416, and U.S. Pat. No. 4,291,071. Cationic softeners can also be used without clay softeners.

### Cationic Polymers

The compositions of the present invention may contain a cationic polymer. Concentrations of the cationic polymer in the composition typically range from about 0.05% to about 3%, in another embodiment from about 0.075% to about 2.0%, and in yet another embodiment from about 0.1% to about 1.0%. Suitable cationic polymers will have cationic charge densities of at least about 0.5 meq/gm, in another embodiment at least about 0.9 meq/gm, in another embodiment at least about 1.2 meq/gm, in yet another embodiment at least about 1.5 meq/gm, but in one embodiment also less than about 7 meq/gm, and in another embodiment less than about 5 meq/gm, at the pH of intended use of the composition, which pH will generally range from about pH 3 to about pH 9, in one embodiment between about pH 4 and about pH 8. Herein, “cationic charge density” of a polymer refers to the ratio of the number of positive charges on the polymer to the molecular weight of the polymer. The average molecular weight of such suitable cationic polymers will generally be between about 10,000 and 10 million, in one embodiment between about 50,000 and about 5 million, and in another embodiment between about 100,000 and about 3 million.

Suitable cationic polymers for use in the compositions of the present invention contain cationic nitrogen-containing moieties such as quaternary ammonium or cationic protonated amino moieties. Any anionic counterions can be used in association with the cationic polymers so long as the polymers remain soluble in water, in the composition, or in a coacervate phase of the composition, and so long as the counterions are physically and chemically compatible with the essential components of the composition or do not otherwise unduly impair product performance, stability or aesthetics. Nonlimiting examples of such counterions include halides (e.g., chloride, fluoride, bromide, iodide), sulfate and methylsulfate.

Other suitable cationic polymers for use in the composition include polysaccharide polymers, cationic guar gum derivatives, quaternary nitrogen-containing cellulose ethers, synthetic polymers, copolymers of etherified cellulose, guar and starch. When used, the cationic polymers herein are either soluble in the composition or are soluble in a complex coacervate phase in the composition formed by the cationic polymer and the anionic, amphoteric and/or zwitterionic surfactant component described hereinbefore. Complex coacervates of the cationic polymer can also be formed with other charged materials in the composition.

Suitable cationic polymers are described in U.S. Pat. Nos. 3,962,418; 3,958,581; and U.S. Publication No. 2007/0207109A1, which are all hereby incorporated by reference.

### Nonionic Polymer

The composition of the present invention may include a nonionic polymer as a conditioning agent. Polyalkylene glycols having a molecular weight of more than about 1000 are useful herein. Useful are those having the following general formula:

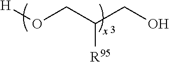

where R⁹⁵ is selected from the group consisting of H, methyl, and mixtures thereof. Conditioning agents, and in particular silicones, may be included in the composition. The conditioning agents useful in the compositions of the present invention typically comprise a water insoluble, water dispersible, non-volatile, liquid that forms emulsified, liquid particles. Suitable conditioning agents for use in the composition are those conditioning agents characterized generally as silicones (e.g., silicone oils, cationic silicones, silicone gums, high refractive silicones, and silicone resins), organic conditioning oils (e.g., hydrocarbon oils, polyolefins, and fatty esters) or combinations thereof, or those conditioning agents which otherwise form liquid, dispersed particles in the aqueous surfactant matrix herein. Such conditioning agents should be physically and chemically compatible with the essential components of the composition, and should not otherwise unduly impair product stability, aesthetics or performance.

The concentration of the conditioning agent in the composition should be sufficient to provide the desired conditioning benefits. Such concentration can vary with the conditioning agent, the conditioning performance desired, the average size of the conditioning agent particles, the type and concentration of other components, and other like factors.

The concentration of the silicone conditioning agent typically ranges from about 0.01% to about 10%. Non-limiting examples of suitable silicone conditioning agents, and optional suspending agents for the silicone, are described in U.S. Reissue Pat. No. 34,584, U.S. Pat. Nos. 5,104,646; 5,106,609; 4,152,416; 2,826,551; 3,964,500; 4,364,837; 6,607,717; 6,482,969; 5,807,956; 5,981,681; 6,207,782; 7,465,439; 7,041,767; 7,217,777; US Patent Application Nos. 2007/0286837A1; 2005/0048549A1; 2007/0041929A1; British Pat. No. 849,433; German Patent No. DE 10036533, which are all incorporated herein by reference; Chemistry and Technology of Silicones, New York: Academic Press (1968); General Electric Silicone Rubber Product Data Sheets SE 30, SE 33, SE 54 and SE 76; Silicon Compounds, Petrarch Systems, Inc. (1984); and in Encyclopedia of Polymer Science and Engineering, vol. 15, 2d ed., pp 204-308, John Wiley & Sons, Inc. (1989).

### Organic Conditioning Oil

The compositions of the present invention may also comprise from about 0.05% to about 3% of at least one organic conditioning oil as the conditioning agent, either alone or in combination with other conditioning agents, such as the silicones (described herein). Suitable conditioning oils include hydrocarbon oils, polyolefins, and fatty esters. Also suitable for use in the compositions herein are the conditioning agents described by the Procter & Gamble Company in U.S. Pat. Nos. 5,674,478, and 5,750,122. Also suitable for use herein are those conditioning agents described in U.S. Pat. Nos. 4,529,586, 4,507,280, 4,663,158, 4,197,865, 4,217,914, 4,381,919, and 4,422,853, which are all hereby incorporated by reference.

### Humectant

The compositions of the present invention may contain a humectant. The humectants herein are selected from the group consisting of polyhydric alcohols, water soluble alkoxylated nonionic polymers, and mixtures thereof. The humectants, when used herein, are preferably used at levels of from about 0.1% to about 20%, more preferably from about 0.5% to about 5%.

### Suspending Agent

The compositions of the present invention may further comprise a suspending agent at concentrations effective for suspending water-insoluble material in dispersed form in the compositions or for modifying the viscosity of the composition. Such concentrations range from about 0.1% to about 10%, preferably from about 0.3% to about 5.0%.

Suspending agents useful herein include anionic polymers and nonionic polymers (e.g., vinyl polymers, acyl derivatives, long chain amine oxides, and mixtures thereof, alkanol amides of fatty acids, long chain esters of long chain alkanol amides, glyceryl esters, primary amines having a fatty alkyl moiety having at least about 16 carbon atoms, secondary amines having two fatty alkyl moieties each having at least about 12 carbon atoms). Examples of suspending agents are described in U.S. Pat. No. 4,741,855.

### Suds Boosters

If high sudsing is desired, suds boosters such as the C₁₀-C₁₆ alkanolamides can be incorporated into the compositions, typically at 1%-10% levels. The C₁₀-C₁₄ monoethanol and diethanol amides illustrate a typical class of such suds boosters. Use of such suds boosters with high sudsing adjunct surfactants such as the amine oxides, betaines and sultaines noted above is also advantageous. If desired, water-soluble magnesium and/or calcium salts such as MgCl₂, MgSO₄, CaCl₂, CaSO₄ and the like, can be added at levels of, typically, 0.1%-2%, to provide additional suds and to enhance grease removal performance.

### Pearlescent Agents

Pearlescent agents as described in WO2011/163457 may be incorporated into the compositions of the invention.

### Perfume

Preferably the composition comprises a perfume, preferably in the range from 0.001 to 3 wt %, most preferably from 0.1 to 1 wt %. Many suitable examples of perfumes are provided in the CTFA (Cosmetic, Toiletry and Fragrance Association) 1992 International Buyers Guide, published by CFTA Publications and OPD 1993 Chemicals Buyers Directory 80^{th} Annual Edition, published by Schnell Publishing Co. It is usual for a plurality of perfume components to be present in the compositions of the invention, for example four, five, six, seven or more. In perfume mixtures preferably 15 to 25 wt % are top notes. Top notes are defined by Poucher (Journal of the Society of Cosmetic Chemists 6(2):80 [1995]). Preferred top notes include rose oxide, citrus oils, linalyl acetate, lavender, linalool, dihydromyrcenol and cis-3-hexanol.

### Other Adjunct Ingredients

A wide variety of other ingredients useful in the cleaning compositions can be included in the compositions herein, including other active ingredients, carriers, hydrotropes, processing aids, dyes or pigments, solvents for liquid formulations, and solid or other liquid fillers, erythrosine, colliodal silica, waxes, probiotics, surfactin, aminocellulosic polymers, Zinc Ricinoleate, perfume microcapsules, rhamnolipds, sophorolipids, glycopeptides, methyl ester sulfonates, methyl ester ethoxylates, sulfonated estolides, cleavable surfactants, biopolymers, silicones, modified silicones, aminosilicones, deposition aids, locust bean gum, cationic hydroxyethylcellulose polymers, cationic guars, hydrotropes (especially cumenesulfonate salts, toluenesulfonate salts, xylenesulfonate salts, and naphalene salts), antioxidants, BHT, PVA particle-encapsulated dyes or perfumes, pearlescent agents, effervescent agents, color change systems, silicone polyurethanes, opacifiers, tablet disintegrants, biomass fillers, fast-dry silicones, glycol distearate, hydroxyethylcellulose polymers, hydrophobically modified cellulose polymers or hydroxyethylcellulose polymers, starch perfume encapsulates, emulsified oils, bisphenol antioxidants, microfibrous cellulose structurants, properfumes, styrene/acrylate polymers, triazines, soaps, superoxide dismutase, benzophenone protease inhibitors, functionalized TiO2, dibutyl phosphate, silica perfume capsules, and other adjunct ingredients, diethylenetriaminepentaacetic acid, Tiron (1,2-dihydroxybenzene-3,5-disulfonic acid), hydroxyethanedimethylenephosphonic acid, methylglycinediacetic acid, choline oxidase, pectate lyase, triarylmethane blue and violet basic dyes, methine blue and violet basic dyes, anthraquinone blue and violet basic dyes, azo dyes basic blue 16, basic blue 65, basic blue 66 basic blue 67, basic blue 71, basic blue 159, basic violet 19, basic violet 35, basic violet 38, basic violet 48, oxazine dyes, basic blue 3, basic blue 75, basic blue 95, basic blue 122, basic blue 124, basic blue 141, Nile blue A and xanthene dye basic violet 10, an alkoxylated triphenylmethane polymeric colorant; an alkoxylated thiopene polymeric colorant; thiazolium dye, mica, titanium dioxide coated mica, bismuth oxychloride, and other actives. Additional ingredients suitable for use in the detergent compositions of the invention are described in U.S. Patent Application No. 2002/0082182A1.

### Fillers and Carriers

An important component of the detergent compositions herein are the fillers and carriers of the composition. It should be noted that the terms “filler” and “carrier”, when used in the specification or in a claim, may be used interchangeably, e.g. any of the following ingredients called a filler may also be considered a carrier.

Liquid detergent compositions, and other detergent forms including a liquid component (such as liquid-containing unit dose detergents), can contain water and other solvents as fillers or carriers. For liquid detergent compositions, the filler or carrier is preferably water. Low molecular weight primary or secondary alcohols exemplified by methanol, ethanol, propanol, and isopropanol are suitable for many liquid detergent applications, especially those in which water is not suitable. Monohydric alcohols are preferred for solubilizing surfactant, but polyols such as those containing from 2 to about 6 carbon atoms and from 2 to about 6 hydroxy groups (e.g., 1,3-propanediol, ethylene glycol, glycerine, and 1,2-propanediol) can also be used. Amine-containing solvents may also be used; suitable amines are described above in the section entitled “amine-neutralized surfactants” and may be used on their own in addition to be used to neutralize acid detergent components. The compositions may contain from 5% to 90%, typically 10% to 50% by weight of such carriers. The isoprenoid-derived surfactants of the present invention are particularly suited for compact or super-compact liquid or liquid-containing detergent compositions. For compact or super-compact heavy duty liquid or other detergent forms, the use of water may be lower than 40%, or lower than 20%, or lower than 5 wt %, or less than 4% or less than 3% free water, or less than 2% free water, or substantially free of free water (i.e. anhydrous).

For powder or bar detergent embodiments, suitable fillers include but are not limited to sodium sulfate, sodium chloride, clay, or other inert solid ingredients. Fillers may also include biomass or decolorized biomass. Typically, fillers in granular, bar, or other solid detergents comprise less than 80 wt %, preferably less than 50 wt %. The isoprenoid-derived surfactants of the present invention are also particularly suited for compact or super-compact powder, solid or powder- or solid-containing detergent compositions. Compact or supercompact powder or solid detergents are included in the present invention, and may involve less than 40%, or less than 20%, or less than 10 wt % filler.

For either compacted or supercompacted liquid detergents or powder detergents, or other detergent forms, the level of liquid or solid filler in the product is reduced, such that either the same amount of active chemistry is delivered to the wash liquor as compared to noncompacted detergents, or more preferably, the cleaning system (surfactants and other adjuncts named herein above) is more efficient such that less active chemistry is delivered to the wash liquor as compared to noncompacted detergents, such as via the use of the novel surfactant system described in the present invention. For example, the wash liquor may be formed by contacting the laundry detergent to water in such an amount so that the concentration of laundry detergent composition in the wash liquor is from above 0 g/1 to about 4 g/l, preferably from 1 g/l to about 3.0 g/l. These dosages are not intended to be limiting, and other dosages may be included in the present invention.

### Buffer System

The cleaning compositions herein will preferably be formulated such that, during use in aqueous cleaning operations, the wash water will have a pH of between about 5.0 and about 12, preferably between about 7.0 and 10.5. Liquid dishwashing product formulations preferably have a pH between about 6.8 and about 9.0. Laundry products are typically at pH 7-11. Techniques for controlling pH at recommended usage levels include the use of buffers, alkalis, acids, etc., and are well known to those skilled in the art. These include the use of sodium carbonate, citric acid or sodium citrate, lactic acid, monoethanol amine or other amines, boric acid or borates, and other pH-adjusting compounds well known in the art.

### Methods of Use

The present invention includes a method for cleaning a targeted surface. As used herein “targeted surface” may include such surfaces such as fabric, dishes, glasses, and other cooking surfaces, hard surfaces. As used herein “hard surface” includes hard surfaces being found in a typical home such as hard wood, tile, ceramic, plastic, leather, metal, glass. Such method includes the steps of contacting the composition of the invention, in neat form or diluted in wash liquor, with at least a portion of a targeted surface then optionally rinsing the targeted surface. Preferably the targeted surface is subjected to a washing step prior to the aforementioned optional rinsing step. For purposes of the present invention, washing includes, but is not limited to, scrubbing, wiping and mechanical agitation.

As will be appreciated by one skilled in the art, the cleaning compositions of the present invention are ideally suited for use in home care (hard surface cleaning compositions) and/or laundry applications.

The compositions are preferably employed at concentrations of from about 200 ppm to about 10,000 ppm in solution. The water temperatures preferably range from about 5° C. to about 100° C.

For use in laundry cleaning compositions, the compositions are preferably employed at concentrations from about 200 ppm to about 10000 ppm in solution (or wash liquor). The water temperatures preferably range from about 5° C. to about 60° C. The water to fabric ratio is preferably from about 1:1 to about 20:1.

The method may include the step of contacting a nonwoven substrate impregnated with an embodiment of the composition of the present invention As used herein “nonwoven substrate” can comprise any conventionally fashioned nonwoven sheet or web having suitable basis weight, caliper (thickness), absorbency and strength characteristics. Examples of suitable commercially available nonwoven substrates include those marketed under the tradename SONTARA® by DuPont and POLYWEB® by James River Corp.

As will be appreciated by one skilled in the art, the cleaning compositions of the present invention are ideally suited for use in liquid dish cleaning compositions. The method for using a liquid dish composition of the present invention comprises the steps of contacting soiled dishes with an effective amount, typically from about 0.5 ml. to about 20 ml. (per 25 dishes being treated) of the liquid dish cleaning composition of the present invention diluted in water.

In addition, another advantage of the highly-branched surfactant-containing systems mixtures and the detergent compositions containing them is their desirable performance in cold water. The invention herein includes methods for laundering of fabrics at reduced wash temperatures. This method of laundering fabric comprises the step of contacting a laundry detergent composition to water to form a wash liquor, and laundering fabric in said wash liquor, wherein the wash liquor has a temperature of above 0° C. to 20° C., preferably to 15° C., or to 10° C. The fabric may be contacted to the water prior to, or after, or simultaneous with, contacting the laundry detergent composition with water.

Machine laundry methods herein typically comprise treating soiled laundry with an aqueous wash solution in a washing machine having dissolved or dispensed therein an effective amount of a machine laundry detergent composition in accord with the invention. By an effective amount of the detergent composition it is meant from 20 g to 300 g of product dissolved or dispersed in a wash solution of volume from 5 to 65 liters, as are typical product dosages and wash solution volumes commonly employed in conventional machine laundry methods.

Hand-washing methods, and combined handwashing with semiautomatic washing machines are also included.

As noted, the mixtures of highly branched surfactant derivatives and optional linear or lightly branched surfactant derivatives of present invention are used herein in cleaning compositions, preferably in combination with other detersive surfactants, at levels which are effective for achieving at least a directional improvement in cleaning performance. In the context of a fabric laundry composition, such “usage levels” can vary depending not only on the type and severity of the soils and stains, but also on the wash water temperature, the volume of wash water and the type of washing machine (e.g., top-loading, front-loading, top-loading, vertical-axis Japanese-type automatic washing machine).

As can be seen from the foregoing, the amount of detergent composition used in a machine-wash laundering context can vary, depending on the habits and practices of the user, the type of washing machine, and the like.

A further method of use of the materials of the present invention involves pretreatment of stains prior to laundering.

Hand dishwashing methods are also included in the present invention.

### Machine Dishwashing Methods

Any suitable methods for machine washing or cleaning soiled tableware, particularly soiled silverware are envisaged. A preferred liquid hand dishwashing method involves either the dissolution of the detergent composition into a receptacle containing water, or by the direct application of the liquid hand dishwashing detergent composition onto soiled dishware.

A preferred machine dishwashing method comprises treating soiled articles selected from crockery, glassware, hollowware, silverware and cutlery and mixtures thereof, with an aqueous liquid having dissolved or dispensed therein an effective amount of a machine dishwashing composition in accord with the invention. By an effective amount of the machine dishwashing composition it is meant from 8 g to 60 g of product dissolved or dispersed in a wash solution of volume from 3 to 10 liters, as are typical product dosages and wash solution volumes commonly employed in conventional machine dishwashing methods.

### Packaging for the Compositions

Commercially marketed executions of the compositions can be packaged in any suitable container including those constructed from paper, cardboard, plastic materials and any suitable laminates. An optional packaging execution is described in European Application No. 94921505.7.

### Fabric Enhancing Softening Compositions

As used herein the term “Fabric Enhancing Composition” includes compositions and formulations designed for enhancing textiles, fabrics, garments and other articles containing a fabric surface. Such compositions include but are not limited to, fabric softening compositions, fabric enhancing compositions, or fabric freshening compositions, and may be of the rinse-added type, the “2-in-1” laundry detergent+fabric enhancer type, or the dryer-added type, and may have a form selected from granular, powder, liquid, gel, paste, bar, single-phase or multi-phase unit dose, fabric treatment compositions, laundry rinse additive, wash additive, post-rinse fabric treatment, ironing aid, delayed delivery formulation, and the like. Such compositions may be used as a pre-laundering treatment, a post-laundering treatment, or may be added during the rinse or wash cycle of the laundering operation. The Fabric Enhancing Compositions formulations of the present invention may be in the form of pourable liquids (under ambient conditions). Such compositions will therefore typically comprise an aqueous carrier, which is present at a levels described above (see “Filler” section).

In other embodiments, the invention relates to fabric softening compositions that include about 0.001 wt % to about 100 wt %, preferably about 0.1 wt % to about 80 wt %., more preferably about 1 wt % to about 25 wt %, by weight of the surfactant system.

### EXAMPLES

The following examples illustrate the present invention. It will be appreciated that other modifications of the present invention within the skill of those in the cleansing and conditioning formulation art can be undertaken without departing from the spirit and scope of this invention. All of the formulations exemplified hereinafter are prepared via conventional formulation and mixing methods unless specific methods are given.

All parts, percentages, and ratios herein are by weight unless otherwise specified. Some components may come from suppliers as dilute solutions. The levels given reflect the weight percent of the active material, unless otherwise specified. The excluded diluents and other materials are included as “Minors”.

In the following examples, AS means alkyl sulfate anionic surfactant, AE means alkyl ethoxylate nonionic surfactant, LAS means linear alkylbenzene sulfonate or branched alkylbenzene sulfonate, AES means alkyl ethoxy sulfate anionic surfactant, AENS means alkyl ethoxy sulfate anionic surfactant with an average of N ethoxylation units per molecule, and APG means alkyl polyglycoside surfactant.

### Example 1

### Granular Laundry Detergents

| | A | B | C | D | E |
|---|---|---|---|---|---|
| Formula | wt % | Wt % | wt % | Wt % | wt % |
| Highly branched surfactant or highly | 20^{b} | 20^{d} | 15^{e} | 15^{f} | 8^{g} |
| branched surfactant blend according to | | | | | |
| the present invention^{a} | | | | | |
| Linear or lightly branched surfactant | 5^{c} | 0 | 3ⁱ | 0 | 3^{h} |
| or co-surfactant; or linear or lightly | | | | | |
| branched surfactant or co-surfactant | | | | | |
| blend according to the present | | | | | |
| invention | | | | | |
| Sodium tripolyphosphate | 0 | 0 | 10 | 15 | 0 |
| Zeolite | 10 | 20 | 0 | 0 | 0 |
| Silicate builder | 10 | 7 | 5 | 0 | 0 |
| Sodium Carbonate | 0 | 20 | 10 | 10 | 20 |
| Diethylene triamine penta acetate | 0 | 1 | 0.5 | 0 | 0 |
| Polyacrylate or polyacrylate/maleate | 1 | 3 | 2 | 0 | 0 |
| Carboxy Methyl Cellulose | 0 | 0 | 1 | 1 | 0 |
| Percarbonate or perborate | 2 | 2 | 2 | 0 | 0 |
| Nonanoyloxybenzenesulfonate, | 1.5 | 1.5 | 0 | 0 | 0 |
| sodium salt | | | | | |
| Tetraacetylethylenediamine | 0 | 0 | 2 | 0 | 0 |
| Zinc Phthalocyanine Tetrasulfonate | 0.005 | | | | |
| Brightener | 1 | 0.8 | 0.8 | 0.5 | 0 |
| MgSO₄ | 0.5 | 1.0 | 0 | 0 | 0 |
| Enzymes (protease, amylase, lipase, | 1.0 | 0.5 | 0.7 | 0.7 | 0 |
| and or cellulases) | | | | | |
| Minors (perfume, dyes, suds | Balance | Balance | Balance | Balance | Balance |
| stabilizers) and fillers | | | | | |

^{a}Surfactant derivatives of EYZ, GYZ, or “A and B” (refering to mixtures of surfactant derivatives of 4,8,12-trimethyltridecan-1-ol and 3-ethyl-7,11-dimethyldodecan-1-ol respectively) as described above.
^{b}blend of surfactants EYZ (alkyl C16 E1.8S form); here and in this and subsequent examples, the terms “surfactants EYZ” or “surfactants GYZ” mean that E and G are either individual hydrophobes structures as shown above in the specification, or blends of two or more hydrophobe structures shown in the list of E and G hydrophobe structures, above in the specification.
^{c}LAS
^{d}80:20 blend of surfactants A and B, wherein YZ is a phenylsulfonate moiety whose phenyl group is derived from benzene chosen from the group consisting of bio-derived benzene (as described in WO 2011/012438A1) or kerosene-derived benzene
^{e}2:1 blend of EYZ (alkyl C16 sulfate form) and surfactant LYZ (alkyl C15 E1.8s form), as defined in US Patent Application Nos. 2011/0171155A1 and 2011/0166370A1, where L is either an individual hydrophobe structure or a blend of two or more hydrophobe structures.
^{f}1:1:1 Blend of EYZ (alkyl sulfate C15 form) and Surfactant A (C16AE3S form) and dihydrophile substituted surfactant (C11AE5 NI form)
^{g}50:50 blend of surfactants A and B, C15-16 AS forms
^{h}LAS whose phenyl group is derived from benzene chosen from the group consisting of bio-derived benzene (as described in WO 2011/012438A1) or kerosene-derived benzene
ⁱLinear C12-14 AE3S

### Example 2

### Granular Laundry Detergents

| | A | B | C | D | E |
|---|---|---|---|---|---|
| Formula | wt % | wt % | wt % | Wt % | Wt % |
| Highly branched surfactant or highly | 15^{a} | 10^{b} | 12^{d} | 10^{e} | 40^{g} |
| branched surfactant blend according to | | | | | |
| the present invention | | | | | |
| Linear or lightly branched surfactant | 0 | 15^{c} | 0 | 10^{f} | 5^{h} |
| or co-surfactant; or linear or lightly | | | | | |
| branched surfactant or co-surfactant | | | | | |
| blend according to the present | | | | | |
| invention | | | | | |
| Sodium tripolyphosphate | 0 | 0 | 10 | 0 | 0 |
| Zeolite | 10 | 20 | 0 | 0 | 0 |
| Sodium Silicate | 10 | 7 | 5 | 0 | 0 |
| Sodium Carbonate | 0 | 20 | 10 | 10 | 20 |
| Diethylene triamine penta acetate | 0 | 1 | 0.5 | 0 | 0 |
| Polyacrylate or polyacrylate/maleate | 0 | 3 | 2 | 0 | 0 |
| Alkoxylated polyamine | 0 | 1 | 1.5 | 0 | 0 |
| Soil Release Polymer | 0.5 | 0.3 | 0 | 0 | 0 |
| Chelant | 0.5 | 0.5 | 2 | 0 | 0 |
| Grease Cleaning Polymer | 1 | 1 | 0 | 0 | 1 |
| Brightener | 1 | 0.8 | 0.8 | 0.5 | 0 |
| Enzymes (protease, amylase, lipase, | 2.0 | 0.5 | 1.0 | 0.7 | 0 |
| and or cellulases) | | | | | |
| Minors (perfume, dyes, suds | Balance | Balance | balance | Balance | balance |
| stabilizers) and fillers | | | | | |

^{a}70-30 blend of surfactants A & B, C16 AE1.1S form
^{b}3:1 blend of surfactants EYZ (C16AS form) and even-scattered branched surfactant LYZ (C12,14,16 blend in AE2S form) as defined in US Patent Application Nos. 2011/0171155A1 and 2011/0166370A1, where L is either an individual hydrophobe structure or a blend of two or more hydrophobe structures
^{c}LAS
^{d}Surfactant EYZ in C16AS form, structures i.-v..
^{e}2:1 blend of surfactant EYZ (C16 AE0.8S form in a mixture of structures i., ii., iv., and v.) and surfactant EYZ (in C15-16 AE7 NI form, structures i.-ii.)
^{f}2:1 blend of LAS and C45E7 NI
^{g}1:1:1 blend of surfactants A (C16AS form), B (C16AS form), and surfactant LYZ form (C12,14,16 alkyl dimethyl monohydroxy ethyl cationic surfactant), as defined in US Patent Application Nos. 2011/0171155A1 and 2011/0166370A1.

### Example 3

### Liquid Laundry Detergents

| | A | B | C | D | E |
|---|---|---|---|---|---|
| Ingredient | Wt % | Wt % | wt % | wt % | wt % |
| Highly branched surfactant or highly | 5^{a} | 10^{b} | 15^{c} | 30^{d} | 30^{e} |
| branched surfactant blend according to | | | | | |
| the present invention | | | | | |
| Linear or lightly branched surfactant or | 10^{f} | 10^{g} | 0 | 0 | 10^{h} |
| co-surfactant; or linear or lightly | | | | | |
| branched surfactant or co-surfactant | | | | | |
| blend according to the present invention | | | | | |
| Citric acid | 2.0 | 3.4 | 1.9 | 1.0 | 1.6 |
| Protease | 1.0 | 0.7 | 1.0 | 0 | 2.5 |
| Amylase | 0.2 | 0.2 | 0 | 0 | 0.3 |
| Lipase | 0 | 0 | 0.2 | 0 | 0 |
| Borax | 1.5 | 2.4 | 2.9 | 0 | 0 |
| Calcium and sodium formate | 0.2 | 0 | 0 | 0 | 0 |
| Formic acid | 0 | 0 | 0 | 0 | 1.1 |
| Ethoxylated polyamine derivative | 1.7 | 2.0 | 0 | 0.8 | 0 |
| polymer or grease cleaning polymers | | | | | |
| Sodium polyacrylate copolymer | 0 | 0 | 0.6 | 0 | 0 |
| DTPA | 0.1 | 0 | 0 | 0 | 0.9 |
| DTPMP | 0 | 0.3 | 0 | 0 | 0 |
| EDTA | 0 | 0 | 0 | 0.1 | 0 |
| Fluorescent whitening agent | 0.15 | 0.2 | 0.12 | 0.12 | 0.2 |
| Ethanol | 2.5 | 1.4 | 1.5 | 0 | 0 |
| Propanediol | 6.6 | 4.9 | 4.0 | 0 | 15.7 |
| Sorbitol | 0 | 0 | 4.0 | 0 | 0 |
| Ethanolamine | 1.5 | 0.8 | 0.1 | 0 | 11.0 |
| Sodium hydroxide | 3.0 | 4.9 | 1.9 | 1.0 | 0 |
| Hydrotropes (sodium cumene sulfonate, | 3.0 | 2.0 | 0 | 0 | 0 |
| sodium toluene sulfonate, sodium | | | | | |
| xylene sulfonate) | | | | | |
| Silicone suds suppressor | 0 | 0.01 | 0 | 0 | 0 |
| Minors (perfume, dyes, opaciefier, | balance | balance | balance | balance | balance |
| adjuncts), water | | | | | |

^{a}60:40 blend of surfactants A and B in C16AE2S form
^{b}1:1 blend of surfactant EYZ (C21AE3S form, structure vi.) and EYZ (C16E2S form, structure i.)
^{c}Synthetic branched surfactants according to U.S. Pat. No. 6,150,322 with greater than 2.4 branches per molecule, in a 1:1 blend of AS and AE2S forms
^{d}90:10 blend of surfactants A and B in C16-18 MES form
^{e}4:4:1 blend of surfactant LYZ (C12-14 sulfonate form), as defined in US Patent Application Nos. 2011/0171155A1 and 2011/0166370A1, surfactant GYZ (wherein G is a C11-isoprenoid-derived alkyl group, structure xii, and YZ is a phenyl sulfonate moiety whose phenyl group is derived from benzene chosen from the group consisting of bio-derived benzene (as described in WO 2011/012438A1) or kerosene-derived benzene), and surfactant EYZ (in C16 dimethyl amine oxide form, structure iii.)
^{f}LAS
^{g}1:1 blend of LAS and Neodol 25-9
^{h}Linear C24 AE3S

### Example 4

### Liquid Laundry Detergents

| | F | G | H | I | J |
|---|---|---|---|---|---|
| Ingredient | Wt % | | | | |
| Highly branched surfactant or highly branched | 40^{a} | 10^{b} | 15^{c} | 20^{d} | 12^{e} |
| surfactant blend according to the present invention | | | | | |
| Linear or lightly branched surfactant or co-surfactant; | 20^{f} | 10^{g} | 3^{h} | 0 | 3ⁱ |
| or linear or lightly branched surfactant or co- | | | | | |
| surfactant blend according to the present invention | | | | | |
| C12-18 fatty acid | 2.6 | 3 | 4 | 0 | 0 |
| Citric acid | 2.6 | 0 | 0 | 2 | 0 |
| Polymer(s) (chosen from the group consisting of | 1 | 1 | 0 | 0 | 0.5 |
| grease cleaning polymer, ethoxylated polyamine | | | | | |
| derivative polymer, modified polyacrylate polymer, | | | | | |
| dye-transfer inhibition polymer, soil release polymer) | | | | | |
| Enzymes - chosen from the group consisting of | 2.0 | 1 | 0.6 | 0.3 | 0 |
| proteases(s), amylase(s), pectate lyase(s), cellulases, | | | | | |
| lipases | | | | | |
| Diethylenetriaminepenta(methylenephosphonic) acid | 0.2 | 0.3 | 0 | 0 | 0.2 |
| Hydroxyethane diphosphonic acid | 0 | 0 | 0.45 | 0 | 0 |
| Brightener | 0.1 | 0.1 | 0.1 | 0 | 0 |
| Solvents (1,2 propanediol, ethanol), stabilizers | 3 | 4 | 1.5 | 1.5 | 2 |
| Structurant | 0.4 | 0.3 | 0.3 | 0.1 | 0.3 |
| Boric acid | 1.5 | 2 | 1 | 0 | 0 |
| Na formate | — | 1 | — | 1 | — |
| Reversible protease inhibitor | — | — | 0.002 | — | — |

| | |
|---|---|
| Buffers (sodium hydroxide, Monoethanolamine, etc), | Balance |
| minors, antifoam, perfume, dyes, water | |

^{a}95:5 blend of surfactants A and B in C15-16 AE2S forms
^{b}Surfactant EYZ, structures i-v in C15 AE9 NI form
^{c}2:1 blend of surfactant EYZ, structures i-v, in C16 AE0.8S form and C16 alkyl sulfonate form
^{d}90:1 A + B blend in C16AE3S form
^{e}Surfactant EYZ, structures i-v, in C16 AS form
^{f}LAS
^{g}1:1 blend of LAS and surfactant GYZ, structures xii-xv, in C11AS form
^{h}LAS
ⁱLinear C24E7 NI

### Example 5

### Liquid Laundry Detergents

| | K | L | M | N | O |
|---|---|---|---|---|---|
| Ingredient | Wt % | | | | |
| Highly branched surfactant or highly | 40^{a} | 20^{b} | 15^{c} | 12^{d} | 20^{e} |
| branched surfactant blend according | | | | | |
| to the present invention | | | | | |
| Linear or lightly branched surfactant | 30^{f} | 3^{g} | 0 | 3^{h} | 0 |
| or co-surfactant; or linear or lightly | | | | | |
| branched surfactant or | | | | | |
| co-surfactant blend according | | | | | |
| to the present invention | | | | | |
| C12-18 fatty acid | 2.2 | 2.0 | — | 1.3 | 2.6 |
| Citric acid | 7 | 0 | 0 | 0 | 2.5 |
| Polymer(s) (chosen from the group | 1.7 | 1.4 | 0.4 | 0 | 0.5 |
| consisting of grease cleaning | | | | | |
| polymer, ethoxylated polyamine | | | | | |
| derivative polymer, modified | | | | | |
| polyacrylate polymer, dye- | | | | | |
| transfer inhibition polymer) | | | | | |
| Enzymes - chosen from the group | 0.4 | 0.3 | 1.0 | 0 | 0 |
| consisting of proteases(s), amylase(s), | | | | | |
| pectate lyase(s), cellulases, lipases | | | | | |
| Chelant(s) | 0.2 | 1.0 | 0 | 0 | 0.2 |
| Solvents (1,2 propanediol, ethanol, | 7 | 7.2 | 3.6 | 3.7 | 1.9 |
| stabilizers | | | | | |
| Structurant | 0.3 | 0.2 | 0.2 | 0.2 | 0.35 |
| Borax | 3 | 3 | 2 | 1.3 | — |
| Boric acid | 1.5 | 2 | 2 | 1.5 | 1.5 |
| Perfume | 0.5 | 0.5 | 0.5 | 0.8 | 0.5 |
| Buffers (sodium hydroxide, | 0.5 | 1 | 2 | 2 | 3.3 |
| monoethanolamine) | | | | | |

| | |
|---|---|
| Water, dyes and miscellaneous | Balance |

^{a}1:1 blend of surfactants EYZ, structures i-v, (in C16AE1S form) and surfactants, structures vi-xi, EYZ (in C21AE3S form)
^{b}50:50 blend of surfactants A and B (in C16AE2S)
^{c}4:1:1 blend of surfactants EYZ, structures i and iii, (in C15-16 AS form), surfactants EYZ, structures i and iv, (in C15E9 NI form), and surfactants EYZ, structures i-v, (in C16 alkyl fatty acid form)
^{d}2:2:1 blend of surfactant A (in AE2S form), surfactant B (in AE2S form), and surfactants LYZ in C12-14 dimethyl amine oxide form, as defined in US Patent Application Nos. 2011/0171155A1 and 2011/0166370A1.
^{e}2:2:1 blend of surfactants EYZ, structures i-v, (in C15-16 AE0.8S form), surfactant A (in C16 alkylpolyglycoside form), and surfactant B (in C16 alkylpolyglycoside form)
^{f}LAS
^{g}Linear C12-14 AE3S
^{h}Surfactants MYZ in C12/14/16 in alkyl sulfate form, as defined in US Patent Application Nos. 2011/0171155A1 and 2011/0166370A1; in this and subsequent examples, the terms “surfactant MZY” or “surfactants MYZ” mean that M is either an individual hydrophobe structure or is a blend of two or more hydrophobe structures.

### Example 6

### Liquid Laundry Detergent

| | P | Q | R |
|---|---|---|---|
| Ingredient | Wt % | | |
| Highly branched surfactant or highly | 10^{a} | 5^{b} | 6^{c} |
| branched surfactant blend according to the | | | |
| present invention | | | |
| Linear or lightly branched surfactant or | 0 | 5^{d} | 2³ |
| co-surfactant; or linear or lightly branched | | | |
| surfactant or co-surfactant blend according | | | |
| to the present invention | | | |

| | |
|---|---|
| Minors (NaOH, buffers, dye, perfume), | Balance to 100 |
| and water | |

^{a}65:35 blend of surfactants A and B in C16AE9 NI form
^{b}Surfactants EYZ, structures i-v, in C16AS form
^{c}4:1 blend of surfactants EYZ, structures iv-v, (in C16AE0.8S form) and surfactants LYZ (in C14 dimethyl amine oxide form), as defined in US Patent Application Nos. 2011/0171155A1 and 2011/0166370A1.
^{d}LAS
^{e}Neodol 23-9

### Example 7

### Liquid Hand Dishwashing Detergents

| | A | B | C |
|---|---|---|---|
| Composition | wt % | wt % | wt % |
| Highly branched surfactant or highly | 20^{a} | 15^{b} | 15^{c} |
| branched surfactant blend according to the | | | |
| present invention | | | |
| Linear or lightly branched surfactant or | 10^{d} | 0 | 2^{e} |
| co-surfactant; or linear or lightly | | | |
| branched surfactant or co-surfactant | | | |
| blend according to the present invention | | | |
| Ethanol | 4 | 0 | 2 |
| Sodium cumene sulfonate | 2.0 | 1.5 | 1 |
| Polypropylene glycol 2000 | 1.0 | 0 | 0 |
| NaCl | 0.8 | 0.8 | 0.4 |
| 1,3 BAC Diamine | 0.5 | 0 | 0 |
| (1,3 bis(methylamine)-cyclohexane) | | | |
| Suds boosting polymer | 0.3 | 0 | 0 |
| ((N,N-dimethylamino)ethyl methacrylate | | | |
| homopolymer) | | | |
| Water | Balance | Balance | Balance |

^{a}3:1 blend of surfactants EYZ, structures i-v, (in C16AE1S form) and surfactants EYZ, structures vi-xi, (in C21AE3S form)
^{b}60:40 blend of surfactants A and B (in C16AE0.8S)
^{c}2:1:1 blend of surfactants EYZ, structures i, ii, iv, and v, (in C16 alkylpolyglycoside form), other isoprenoid surfactants (in C11 disulfonate form), other isoprenoid surfactants (C16 disulfonate form)
^{d}LAS
^{e}Surfactants GYZ, structures xii-xv, in C11AS form

### Example 8

### Powder, Liquid, Tablet, Unit Dose, or Gel Automatic Dishwasher Detergents

| | A | B | C | D | E |
|---|---|---|---|---|---|
| | wt % | wt % | wt % | wt % | wt % |
| Highly branched | 3^{a} | 3^{b} | 1^{c} | 1^{d} | 1.5^{e} |
| surfactant or highly | | | | | |
| branched surfactant | | | | | |
| blend according | | | | | |
| to the present invention | | | | | |
| Linear or lightly | 0 | 0 | 1^{f} | 1^{g} | 3^{h} |
| branched surfactant or | | | | | |
| co-surfactant; or linear or | | | | | |
| lightly branched | | | | | |
| surfactant or | | | | | |
| co-surfactant | | | | | |
| blend according to the | | | | | |
| present invention | | | | | |
| Suds Supressor | 0 | 0 | 3 | 3 | 0 |
| Surfactant (low cloud | | | | | |
| point NI, such as | | | | | |
| SLF18, LF404) | | | | | |
| Polymer (chosen from | 1 | 0 | 2 | 3 | 0 |
| among the group of | | | | | |
| polyacrylate, | | | | | |
| polyacrylate | | | | | |
| maleate, modified | | | | | |
| polyacrylate maleate, | | | | | |
| polyacrylate- | | | | | |
| methyacrylate) | | | | | |
| Sodium Carbonate | 35 | 10 | 0 | 35-40 | 35-40 |
| Sodium tripolyphosphate | 0 | 20 | 10 | 0 | 0-10 |
| Silicate solids | 6 | 6 | 0 | 1 | 0 |
| Bleaching system | 0-4 | 0-4 | 0 | 0 | 0-4 |
| (Chosen from | | | | | |
| among the group | | | | | |
| consisting of NaDCC, | | | | | |
| perborate, percarbonate, | | | | | |
| NaOCl, transition metal | | | | | |
| catalyst) | | | | | |
| Polymer Thickener | 0 | 1 | | | |
| Enzymes | 0.3-0.6 | 0.3-0.6 | 0.3-0.6 | 0.3-0.6 | 0.3-0.6 |
| Disodium citrate | 0 | 0 | 0 | 2-20 | 0 |
| dehydrate | | | | | |
| Fillers (water or sulfate) | Balance | Balance | Balance | Balance | Balance |
| and minors (perfume, | to 100% | to 100% | to 100% | to 100% | to 100% |
| dyes and other adjuncts) | | | | | |

^{a}low cloud point NI having a structure in which either SLF18 or LF404 have had their hydrophobes substituted by isoprenoid hydrophobes E, structures i-v, i.e. surfactants EYZ wherein Z is a PO3-EO12-PO15 moiety, where PO is propylene oxide and EO is ethylene oxide
^{b}50:50 blend of surfactants A and B (wherein Z is a PO3-EO12-PO15 moiety, where PO is propylene oxide and EO is ethylene oxide)
^{c}10:90 blend of surfactant A and B (in C16AS form)
^{d}Surfactants EYZ, structures i-v, in C16 dimethyl amine oxide form
^{e}Surfactants LYZ in C12-14E7 NI form, as defined in US Patent Application Nos. 2011/0171155A1 and 2011/0166370A1.
^{f}LAS
^{g}Surfactants GYZ, structure xii, in C11 dimethyl amine oxide form
^{h}surfactants GYZ, structures xii-xv, wherein Z is a PO3-EO12-PO15 moiety, where PO is propylene oxide and EO is ethylene oxide

### Example 9

### Hard Surface Cleaner

| | A | B | C | D | E |
|---|---|---|---|---|---|
| | wt % | wt % | wt % | wt % | wt % |
| Highly branched | 5^{a} | 1^{c} | 3^{d} | 5^{f} | 5^{g} |
| surfactant or highly | | | | | |
| branched surfactant | | | | | |
| blend according | | | | | |
| to the present invention | | | | | |
| Linear or lightly | 5^{b} | 0 | 6^{e} | 0 | 0 |
| branched surfactant or | | | | | |
| co-surfactant; or linear or | | | | | |
| lightly branched | | | | | |
| surfactant or | | | | | |
| co-surfactant | | | | | |
| blend according to the | | | | | |
| present invention | | | | | |
| Inorganic cleaning agents | 0 | 0-40 | 10-20 | 0-2 | 5 |
| (chosen from among the | | | | | |
| group consisting of citric | | | | | |
| acid, sodium | | | | | |
| polyphosphate, | | | | | |
| sodium silicate, sodium | | | | | |
| carbonate) | | | | | |
| Solvents | 0-10 | 0-20 | 0-20 | 0-20 | 0-20 |
| Fillers and minors | Balance | Balance | Balance | Balance | Balance |
| (perfume, dyes and | to 100% | to 100% | to 100% | to 100% | to 100% |
| other adjuncts) | | | | | |

^{a}Surfactants EYZ, structures vi-vii, in C21E6S form
^{b}LAS
^{c}Surfactants EYZ, structures i-v, in C16AE0.9S form
^{d}Surfactants LYZ C12/14 alkylpolyglycoside form, as defined in US Patent Application Nos. 2011/0171155A1 and 2011/0166370A1.
^{e}Neodol 23-9
^{f}65:35 blend of surfactants A and B in C16AS form
^{g}1:1:1 blend of surfactant A (in C16 AE1S form), surfactant B (in C16 AE1S form), and C11 isoprenoidsurfactant, structures xii-xv, where Z is sulfate

### Example 10

### Fabric Softener Compositions

| | Weight %'s | | | |
|---|---|---|---|---|
| Ingredient | A | B | C | D |
| Fabric softener cationic surfactant (T-U)₂V of the present invention | 20^{a} | 30^{b} | 10^{c} | 5^{d} |
| Fabric softener cationic surfactant | 0 | 0 | 10^{e} | 10^{f} |
| Hydrochloric acid | 0.1 | 0.1 | 0 | 0.1 |
| Silicone-based antifoam | 0.5 | 0.3 | 0.5 | 1.0 |
| CaCl2 | 2.0 | 1.0 | 0.5 | 0.5 |
| Soil release polymer | 0.3 | 0 | 0 | 0 |
| Ammonium chloride | 0.5 | 1 | 0 | 0 |
| Perfume, dye, minors, water | Balance | balance | Balance | Balance |

^{a}A blend of di-isoprenoid cationic surfactants having the following structures, wherein the overall ratio of 4,8,12-trimethyltridecan-1-oyl moiety to 3-ethyl-7,11-
dimethyldodecan-1-oyl moieties is greater than about 80:20. 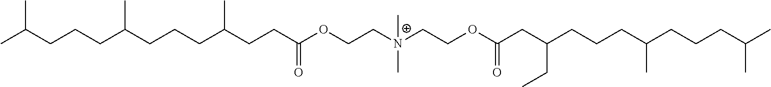 [SMILES: CCC(CCCC(C)CCCC(C)C)CC(=O)OCC[N+](C)(C)CCOC(=O)CCC(C)CCCC(C)CCCC(C)C] 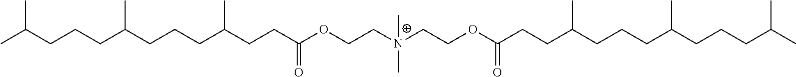 [SMILES: CC(C)CCCC(C)CCCC(C)CCC(=O)OCC[N+](C)(C)CCOC(=O)CCC(C)CCCC(C)CCCC(C)C] 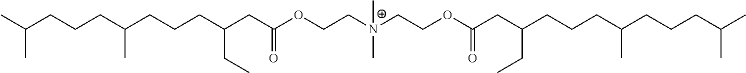 [SMILES: CCC(CCCC(C)CCCC(C)C)CC(=O)OCC[N+](C)(C)CCOC(=O)CC(CC)CCCC(C)CCCC(C)C]
^{b}cationic surfactant T₂N(Me)₂Cl, wherein T is one or more isoprenoid hydrophobes as described in the specification above
^{c}cationic surfactant T₂N(Me)₂Cl, wherein T is a 90:10 mixture of 4,8,12-trimethyltridecan-1-yl and 3-ethyl-7,11-dimethyldodecan-1-yl moeities
^{d}cationic surfactant (TCO₂CH₂CH₂)₂N(Me)₂Cl, wherein T is one or more isoprenoid hydrophobes as described in the specification above
^{e}Dimethyl Bis(Steroyl oxyethyl) ammonium chloride
^{f}Distearyldimethylammonium chloride

### Example 11

### Comparison of Compositions of the Present Invention—Laundry Applications

To demonstrate the superiority of the surfactant system of the present invention, the following four formulas are prepared and tested for DIFT—Dynamic Oil-Water Interfacial Tension, which is an accepted fundamental measure which predicts grease cleaning effectiveness.

### Results

The surfactant systems in Table 3 are analyzed via DIFT method. Ingredients listed are in ppm concentration as would be common in a detergent wash water solution. Analysis conditions are in water of 103 ppm Calcium/Magnesium water hardness level (3:1 Calcium:Magnesium), 21° C., pH 8-8.5.
Formula E contains a commercial branched Neodol® 67 AS surfactant.
Formula F contains 90A:10B AS of the present invention substituted for branched Neodol® 67 AS surfactant.
Formulas G and H are common detergent surfactant mixtures containing no branched alkyl sulfate surfactants.

TABLE 3

| | Formula E | Formula F | Formula G | Formula H |
|---|---|---|---|---|
| AES | 54 ppm | 54 ppm | 167 ppm | 90 ppm |
| LAS | 22 ppm | 22 ppm | 22 ppm | 130 ppm |
| Surfonic ® 24-9 | 7 ppm | 7 ppm | 7 ppm | 120 ppm |
| Neodol ® 67 AS | 167 ppm | | | |
| 90A:10B AS⁹ | | 167 ppm | | |
| Anti-redeposition | 14 ppm | 14 ppm | 14 ppm | |
| agents¹⁰ | | | | |
| Alkoxylated | 13 ppm | 13 ppm | 13 ppm | |
| Polyamine | | | | |
| Polymers¹¹ | | | | |

1. 90A:10B AS is comprised of a mixture of 90 wt % of the sodium sulfate of 4,8,12-trimethyltridecan-1-ol and 10 wt % of the sodium sulfate of 3-ethyl-7,11-dimethyldodecan-1-ol as previously described.
2. Clay Soil/Anti-Redeposition Agents as described under Detailed Description of the Invention.
3. Alkoxylated Polyamine Polymers as described under Detailed Description of the Invention.

TABLE 4
DIFT Measure (mN/m) at 1 uL/min Canola Oil Flowrate

| | Formula E | Formula F | Formula G | Formula H |
|---|---|---|---|---|
| IFT @ 1 uL/min | 0.51 | 0.46 | 1.05 | 2.09 |
| Oil Flowrate (mN/m) | | | | |

As can be seen in Table 4, Formula F containing the 90A:10B AS surfactant, has improved IFT compared to Formula E containing a commercial branched Neodol® 67 surfactant as well as Formulas G and H which contain no branched alkyl sulfate surfactants.

### Method: Dynamic Interfacial Tension Analysis.

Dynamic Interfacial Tension (DIFT) analysis is performed on a Krüss® DVT30 Drop Volume Tensiometer (Krüss USA, Charlotte, N.C.). The instrument is configured to measure the interfacial tension (IFT) of an ascending oil drop in aqueous detergent (surfactant) phase. The oil used is canola oil (Crisco Pure Canola Oil manufactured by The J.M. Smucker Company). The aqueous detergent and oil phases are temperature controlled at 22° C. (+/−1° C.), via a recirculating water temperature controller attached to the tensiometer. A dynamic interfacial tension curve is generated by dispensing the oil drops into the aqueous detergent phase from an ascending capillary with an internal diameter of 0.2540 mm, over a range of flow rates and measuring the interfacial tension at each flow rate. Data is generated at oil dispensing flow rates of 500 uL/min to 1 uL/min with 2 flow rates per decade on a logarithmic scale (7 flow rates measured in this instance). Interfacial tension is measured on three oil drops per flow rate and then averaged. Interfacial tension is reported in units of mN/m. Surface age of the oil drops at each flow rate is also recorded and plots can be generated either of interfacial tension (y-axis) versus oil flow rate (x-axis) or interfacial tension (y-axis) versus oil drop surface age (x-axis). Minimum IFT (mN/m) for an experiment is recorded as the IFT at the slowest flow rate (1 uL/minute as an example), with lower IFT values indicating superior performance. In addition, IFT at higher oil flow rates such as 10 uL/min and 99 uL/min, as example, correspond to shorter surface ages of the oil drops and are an indication of how effective a detergent system is at lowering IFT values at shorter time periods versus longer time periods associated with equilibrium IFT, with lower IFT values again indicating superior performance. Example of analysis of a 100 ppm surfactant concentration, with water hardness (3:1 Ca:Mg) of 103 ppm, 22° C., pH 8:Density settings for 22° C. are set at 0.916 g/ml for Canola Oil and 0.998 g/ml for aqueous surfactant phase (assumed to be the same as water since dilute solution). To a 100 ml volumetric flask is added 1.00 mL of 1% (wt/wt) Surfactant solution in deionized water and the volumetric is then filled to the mark with 108 ppm 3:1 CaCl2:MgCl2 solution and mixed well. The solution is transferred to a beaker and the pH adjusted to 8 by addition of a few drops of 0.1N NaOH or 0.1N H2SO4. The solution is then loaded into the tensiometer measurement cell and analyzed. Total time from addition of hardness to surfactant and start of analysis is less than 5 minutes.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A detergent composition comprising
A. a surfactant system comprising greater than about 25% of one or more highly branched surfactants; and
B. one or more adjunct cleaning additives; and
C. a carrier.

2. The detergent composition of claim 1, wherein the surfactant system further comprises one or more linear or lightly branched surfactants.

3. The detergent composition of claim 1, wherein said surfactant system comprises greater than about 30% of said one or more highly branched surfactants, wherein said one or more highly branched surfactants comprises an isoprenoid-based surfactant of the structure
E-Y-Z
wherein E is one or more saturated, acyclic C10-C24 isoprenoid-based hydrophobe(s), Y is CH₂ or null, and Z is selected such that the resulting surfactant is an alkyl carboxylate surfactant, an alkyl polyalkoxy surfactant, an alkyl anionic polyalkoxy sulfate surfactant, an alkyl glycerol ester sulfonate surfactant, an alkyl dimethyl amine oxide surfactant, an alkyl polyhydroxy based surfactant, an alkyl phosphate ester surfactant, an alkyl glycerol sulfonate surfactant, an alkyl polygluconate surfactant, an alkyl polyphosphate ester surfactant, an alkyl phosphonate surfactant, an alkyl polyglycoside surfactant, an alkyl monoglycoside surfactant, an alkyl diglycoside surfactant, an alkyl sulfosuccinate surfactant, an alkyl disulfate surfactant, an alkyl disulfonate surfactant, an alkyl sulfosuccinamate surfactant, an alkyl glucamide surfactant, an alkyl taurinate surfactant, an alkyl sarcosinate surfactant, an alkyl glycinate surfactant, an alkyl isethionate surfactant, an alkyl dialkanolamide surfactant, an alkyl monoalkanolamide surfactant, an alkyl monoalkanolamide sulfate surfactant, an alkyl diglycolamide surfactant, an alkyl diglycolamide sulfate surfactant, an alkyl glycerol ester surfactant, an alkyl glycerol ester sulfate surfactant, an alkyl glycerol ether surfactant, an alkyl glycerol ether sulfate surfactant, alkyl methyl ester sulfonate surfactant, an alkyl polyglycerol ether surfactant, an alkyl polyglycerol ether sulfate surfactant, an alkyl sorbitan ester surfactant, an alkyl ammonioalkanesulfonate surfactant, an alkyl amidopropyl betaine surfactant, an alkyl allylated quat based surfactant, an alkyl monohydroxyalkyl-di-alkylated quat based surfactant, an alkyl di-hydroxyalkyl monoalkyl quat based surfactant, an alkylated quat surfactant, an alkyl trimethylammonium quat surfactant, an alkyl polyhydroxalkyl oxypropyl quat based surfactant, an alkyl glycerol ester quat surfactant, an alkyl glycol amine quat surfactant, an alkyl monomethyl dihydroxyethyl quaternary ammonium surfactant, an alkyl dimethyl monohydroxyethyl quaternary ammonium surfactant, an alkyl trimethylammonium surfactant, an alkyl imidazoline-based surfactant, an alken-2-yl-succinate surfactant, an alkyl α-sulfonated carboxylic acid surfactant, an alkyl α-sulfonated carboxylic acid alkyl ester surfactant, an alpha olefin sulfonate surfactant, an alkyl phenol ethoxylate surfactant, an alkyl benzenesulfonate surfactant, an alkyl sulfobetaine surfactant, an alkyl hydroxysulfobetaine surfactant, an alkyl ammoniocarboxylate betaine surfactant, an alkyl sucrose ester surfactant, an alkyl alkanolamide surfactant, an alkyl di(polyoxyethylene)monoalkyl ammonium surfactant, an alkyl mono(polyoxyethylene)dialkyl ammonium surfactant, an alkyl benzyl dimethylammonium surfactant, an alkyl aminopropionate surfactant, an alkyl amidopropyl dimethylamine surfactant, or a mixture thereof; and one or more linear or lightly branched surfactants.

4. The detergent composition of claim 3, where said isoprenoid-based surfactant comprises one or more of the surfactants represented by formulas i through xi:

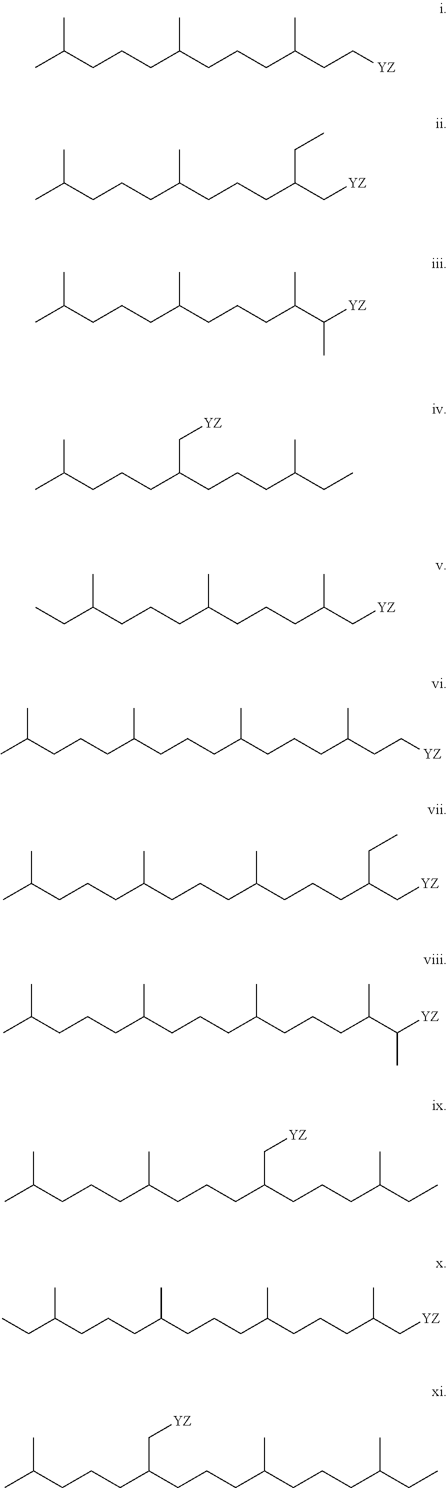

5. The detergent composition of claim 4 wherein said isoprenoid-based surfactant comprises one or more of the surfactants represented by formulas A and B:

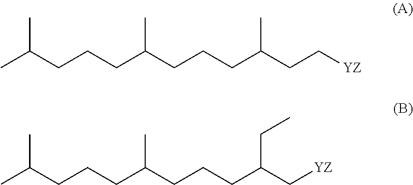

6. The composition of claim 5 wherein the weight ratio of surfactant of formula A to surfactant of formula B is from about 50:50 to about 95:5.

7. The detergent composition of claim 1, wherein said one or more adjunct cleaning additives is selected from a builder, an organic polymeric compound, an enzyme, an enzyme stabilizer, a bleach system, a brightener, a hueing agent, a chelating agent, a suds suppressor, a conditioning agent, a humectant, a perfume, a filler or carrier, an alkalinity system, a pH control system, a buffer, or a mixture thereof.

8. The detergent composition of claim 1, wherein said detergent composition is in the form of a granular detergent, a bar-form detergent, a liquid laundry detergent, a gel detergent, a single-phase or multi-phase unit dose detergent, a detergent contained in a single-phase or multi-phase or multi-compartment water soluble pouch, a liquid hand dishwashing composition, a laundry pretreat product, a detergent contained on or in a porous substrate or nonwoven sheet, a automatic dish-washing detergent, a hard surface cleaner, a hair-care conditioning composition, a, or a fabric softener composition.

9. The detergent composition of claim 1 wherein said detergent composition comprises from about from about 0.1 wt % to about 80 wt % of said surfactant system.

10. The detergent composition of claim 9 wherein the composition comprises from about 5% to about 50% of the surfactant system.

11. The detergent composition of claim 2 wherein said linear or lightly branched surfactant is selected from a C₁₀-C₁₆ alkyl benzene sulfonate, a alkyl sulfate, a alkyl ethoxy sulfate, a alkyl ethoxylates, or a mixture thereof.

12. The composition of claim 6 wherein said adjunct cleaning additive comprises an enzyme.

13. The composition of claim 2 wherein said linear or lightly branched surfactant comprises one or more of the surfactants represented by formulas xii through xv: 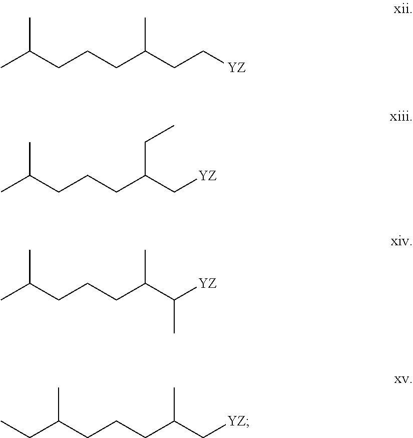
wherein Y is CH₂ or null and Z is selected such that the resulting surfactant is an alkyl carboxylate surfactant, an alkyl polyalkoxy surfactant, an alkyl anionic polyalkoxy sulfate surfactant, an alkyl glycerol ester sulfonate surfactant, an alkyl dimethyl amine oxide surfactant, an alkyl polyhydroxy based surfactant, an alkyl phosphate ester surfactant, an alkyl glycerol sulfonate surfactant, an alkyl polygluconate surfactant, an alkyl polyphosphate ester surfactant, an alkyl phosphonate surfactant, an alkyl polyglycoside surfactant, an alkyl monoglycoside surfactant, an alkyl diglycoside surfactant, an alkyl sulfosuccinate surfactant, an alkyl disulfate surfactant, an alkyl disulfonate surfactant, an alkyl sulfosuccinamate surfactant, an alkyl glucamide surfactant, an alkyl taurinate surfactant, an alkyl sarcosinate surfactant, an alkyl glycinate surfactant, an alkyl isethionate surfactant, an alkyl dialkanolamide surfactant, an alkyl monoalkanolamide surfactant, an alkyl monoalkanolamide sulfate surfactant, an alkyl diglycolamide surfactant, an alkyl diglycolamide sulfate surfactant, an alkyl glycerol ester surfactant, an alkyl glycerol ester sulfate surfactant, an alkyl glycerol ether surfactant, an alkyl glycerol ether sulfate surfactant, alkyl methyl ester sulfonate surfactant, an alkyl polyglycerol ether surfactant, an alkyl polyglycerol ether sulfate surfactant, an alkyl sorbitan ester surfactant, an alkyl ammonioalkanesulfonate surfactant, an alkyl amidopropyl betaine surfactant, an alkyl allylated quat based surfactant, an alkyl monohydroxyalkyl-di-alkylated quat based surfactant, an alkyl di-hydroxyalkyl monoalkyl quat based surfactant, an alkylated quat surfactant, an alkyl trimethylammonium quat surfactant, an alkyl polyhydroxalkyl oxypropyl quat based surfactant, an alkyl glycerol ester quat surfactant, an alkyl glycol amine quat surfactant, an alkyl monomethyl dihydroxyethyl quaternary ammonium surfactant, an alkyl dimethyl monohydroxyethyl quaternary ammonium surfactant, an alkyl trimethylammonium surfactant, an alkyl imidazoline-based surfactant, an alken-2-yl-succinate surfactant, an alkyl α-sulfonated carboxylic acid surfactant, an alkyl α-sulfonated carboxylic acid alkyl ester surfactant, an alpha olefin sulfonate surfactant, an alkyl phenol ethoxylate surfactant, an alkyl benzenesulfonate surfactant, an alkyl sulfobetaine surfactant, an alkyl hydroxysulfobetaine surfactant, an alkyl ammoniocarboxylate betaine surfactant, an alkyl sucrose ester surfactant, an alkyl alkanolamide surfactant, an alkyl di(polyoxyethylene)monoalkyl ammonium surfactant, an alkyl mono(polyoxyethylene)dialkyl ammonium surfactant, an alkyl benzyl dimethylammonium surfactant, an alkyl aminopropionate surfactant, an alkyl amidopropyl dimethylamine surfactant, or a mixture thereof.

14. The composition of claim 1 wherein the resulting pH of the detergent in water during use is lower than 10.

15. The composition of claim 1, further comprising an electrolyte.

16. The composition of claim 1 wherein the composition contains solid or liquid fillers or carriers in the amount of less than 50%.

17. A method of treating a surface with the detergent composition of claim 1 comprising the steps of contacting said composition with water to form a wash liquor and then contacting said surface with said wash liquor.

18. A method according to claim 20, wherein said wash liquor has a temperature from about above 0° C. to about 20° C.

19. The composition of claim 1, wherein said surfactant system includes one or more surfactants selected from near-terminal branched surfactants, di-hydrophile substituted isoprenoid derived surfactants, even scattered branched surfactants, or a mixture thereof.

20. The detergent composition of claim 1, wherein said highly or lightly branched surfactant comprises a surfactant of the following structure:
(T-U)ⱼV
wherein V is a polyhydroxy moiety, a sucrose moiety, a mono-, di-, oligo-, or polysaccharide moiety, a polyglycerol moiety, a dialkyl ammonium moiety, a dimethylammonium moiety, or a gemini surfactant spacer moiety;
U is either absent or is selected from —CO₂—, —CO₂CH₂CH₂—, or a gemini surfactant polar or charged moiety, wherein if either U or V is a charged moiety, the charged moiety is charge balanced by a suitable counterion; j ranges from 2 to 10; and T is one or more isoprenoid-based hydrophobe radicals represented by formulas i through xv: 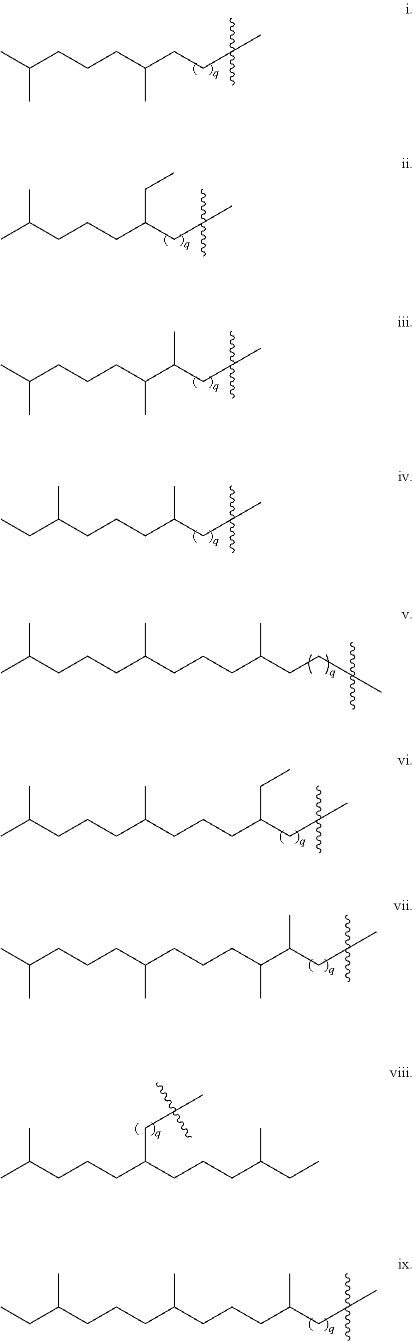 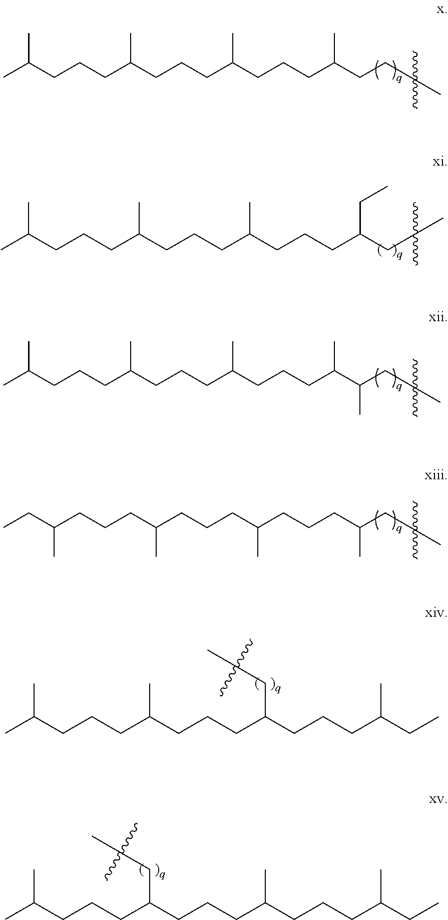
wherein q is 0-5.
